(19) United States
(12) Patent Application Publication
(10) Pub. No.: US 2022/0378565 A1
(43) Pub. Date: Dec. 1, 2022

(54) SYSTEMS AND METHODS FOR TRANSCATHETER AORTIC VALVE TREATMENT

(71) Applicant: Silk Road Medical, Inc., Sunnyvale, CA (US)

(72) Inventors: Stewart M. Kume, Sunnyvale, CA (US); Sumaira MacDonald, Sunnyvale, CA (US); Erica J. Rogers, Sunnyvale, CA (US); Harold Carrison, Sunnyvale, CA (US)

(21) Appl. No.: 17/773,206

(22) PCT Filed: Nov. 2, 2020
(86) PCT No.: PCT/US20/58588

§ 371 Date: Apr. 29, 2022

Related U.S. Application Data
(60) Provisional application No. 62/929,357, filed on Nov. 1, 2019.
(60) Provisional application No. 63/014,979, filed on Apr. 24, 2020.
(60) Provisional application No. 63/039,101, filed on Jun. 15, 2020.

(51) Int. Cl. A61F 2/01; A61F 2/24

(52) U.S. Cl. CPC A61F 2/013; A61F 2/2436; A61F 2/011; A61F 2/0105

(57) ABSTRACT

Devices and methods are configured to allow transcarotid or subclavian access via the common carotid artery to the native aortic valve, and implantation of a prosthetic aortic valve into the heart. The devices and methods also provide for embolic protection during such an endovascular aortic valve implantation procedure.

### CROSS-REFERENCE TO RELATED APPLICATIONS

This application claims priority to U.S. Patent Application No. 62/929,357, filed Nov. 1, 2019, entitled “SYSTEMS AND METHODS FOR TRANSCATHETER AORTIC VALVE TREATMENT”, U.S. Patent Application No. 63/014,979, filed Apr. 24, 2020, entitled “SYSTEMS AND METHODS FOR TRANSCATHETER AORTIC VALVE TREATMENT”, U.S. Patent Application No. 63/039,101, filed Jun. 15, 2020, entitled “SYSTEMS AND METHODS FOR TRANSCATHETER AORTIC VALVE TREATMENT”, the contents of which are hereby incorporated by reference herein in their entirety.

### BACKGROUND

The present disclosure relates to methods and devices for replacing or treating heart valves.

Patients with defective aortic heart valves are often candidates for a replacement heart valve procedure. The conventional treatment is the surgical replacement of the heart valve with a prosthetic valve. This surgery involves a gross thorocotomy or median sternotomy, cardiopulmonary bypass and cardiac arrest, surgical access and excision of the diseased heart valve, and replacement of the heart valve with a prosthetic mechanical or tissue valve. Valves implanted in this manner have historically provided good long term outcomes for these patients, with durability of up to ten or fifteen years for tissue valves, and even longer for mechanical valves. However, heart valve replacement surgery is highly invasive, can require lengthy recovery time, and is associated with short and long term complications. For high surgical risk or inoperable patients, this procedure may not be an option.

Minimally invasive approaches to heart valve replacement has been developed. This approach, known as transcatheter aortic valve implantation (TAVI) or replacement (TAVR), relies on the development of a collapsible prosthetic valve which is mounted onto a catheter-based delivery system. This type of prosthesis can be inserted into the patient through a relatively small incision or vascular access site, and may be implanted on the beating heart without cardiac arrest. The advantages of this approach include less surgical trauma, faster recovery time, and lower complication rates. For high surgical risk or inoperable patients, this approach offers a good alternative to conventional surgery. Examples of this technology are the Sapien Transcatheter Valve (Edwards Lifesciences, Irvine, Calif.) and the CoreValve System (Medtronic, Minneapolis, Minn.). U.S. Pat. No. 6,454,799, which is incorporated herein by reference in its entirety, describes examples of this technology.

There are two main pathways for valves inserted using the TAVI approach. The first is a vascular approach via the femoral artery (referred to as a transfemoral approach), either percutaneously or through a surgical cut-down and arteriotomy of the femoral artery. Once placed into the femoral artery, the valve mounted on the delivery system is advanced in a retrograde manner (in the reverse direction as blood flow) up the descending aorta, around the aortic arch, and across the ascending aorta in order to be positioned across the native aortic valve. Transfemoral aortic valve delivery systems are typically over 90 cm in length and require the ability to navigate around the aortic arch. The relatively small diameter of the femoral artery and the frequent presence of atherosclerotic disease in the iliofemoral anatomy limits the maximum diameter of the delivery system to about 24 French (0.312″) in diameter. The second pathway, termed transapical, involves accessing the left ventricle through the apex of the heart via a mini-thorocotomy, and advancing the valve delivery system in an antegrade fashion (in the same direction as blood flow) to the aortic valve position. This pathway is much shorter and straighter than the transfemoral path, but involves a surgical puncture and subsequent closure of the wall of the heart.

Other approaches have been described, including access from the subclavian artery, and direct puncture of the ascending aorta via a mini-thorocotomy. The subclavian approach (transsubclavian approach) has been used when the transfemoral route is contra-indicated, but may block flow to the cerebral vessel through the ipsilateral common carotid artery. A direct aortic puncture is usually considered if all other routes must be excluded due to anatomic difficulties including vascular disease. Puncture of the aortic wall, and subsequent closure, carries associated surgical risk including aortic dissection and rupture.

The transfemoral approach to the aortic valve, as opposed to the transapical or other alternative approaches, is a generally more familiar one to the medical community. Accessing the ascending aorta from the femoral artery is standard procedure for interventional cardiologists. Balloon valvuloplasty procedures via the transfemoral approach have been performed for years. The surgical approaches such as the transapical access or direct aortic puncture are less familiar and require practitioners with both surgical and endovascular skills; techniques for the surgical approaches are still evolving and whether they offer advantages over the transfemoral and transsubclavian methods have yet to be determined. However, problems also exist with the transfemoral and transsubclavian approaches. One is that the desired access vessel is often too small and/or is burdened with atherosclerotic disease, which precludes the artery as an access point. A second problem is that the pathway from the access point to the aortic valve usually involves one or more major turns of at least 90° with a relatively tight radii of curvature, 0.5″ or less, requiring a certain degree of flexibility in the delivery system. This flexibility requirement restricts the design parameters of both the valve and the delivery system, and together with the required length of the delivery system reduces the level of control in accurately positioning the valve.

Both the transfemoral and transapical approaches have as potential complications the dislodgement of atherosclerotic and/or thrombotic debris, so-called “embolization” or the creation of “embolic debris,” during access maneuvers, pre-dilation of the diseased valve, and implantation of the prosthetic valve. The most serious consequence of embolic debris is that it travels with the blood flow to the brain via one or more of the four primary conduits to the cerebral circulation, namely the right and left carotid arteries and the right and left vertebral arteries. Transfemoral TAVI procedures require passage of large device and delivery system components through the aortic arch and across the origins of the head and neck vessels that supply blood flow to the carotid and vertebral arteries, potentially loosening, fragmenting, and dislodging debris during its route to the aortic valve. The transapical TAVI procedure involves a puncture of the heart wall, which may generate embolic debris from the wall of the ventricle or ascending aorta, or may form thrombus or clot at the apical puncture location. During the vigorous motion of the beating heart, this clot can break free and travel to the brain as well. Both approaches require significant manipulation while the prosthetic valve is being placed: the TAVI implant and delivery system moves back and forth across the native aortic valve, potentially dislodging more debris from the diseased valve itself. With expansion of the valve implant, the native aortic valve is compressed and moved out of the stream of the cardiac output, another moment when the shearing and tearing of the native valve can free more debris to embolize to the brain.

Recently, there has been described an embolic filter protection device for use with TAVI procedures, as referenced in U.S. Pat. No. 8,460,335, which is incorporated herein by reference in its entirety. This device places a temporary screen over the ostium of the head and neck vessels to prevent passage of embolic particles while allowing blood flow into the vessels. While this device may offer some protection from larger embolic particles, it requires an additional vascular access and device deployment, adding to the cost and time of the procedure, and does not facilitate the passage of the prosthetic valve itself. Moreover, it does not provide protection during filter placement and retrieval; since the filter is deployed against the wall of the aorta, there is a high chance that the filter manipulation itself will be the cause of embolic complications.

### SUMMARY

There is a need for an access system for endovascular prosthetic aortic valve implantation that provides a generally shorter and straighter access path than current systems and methods. This would allow the use of shorter and more rigid delivery systems which would offer a greater degree of control and easier placement of the aortic valve. There is also a need for an access system that provides protection from cerebral embolic complications during the procedure.

Disclosed herein are devices and methods that allow transcarotid or subclavian access via the common carotid artery to the native aortic valve, and transcatheter implantation of a prosthetic aortic valve into the heart or aorta. The devices and methods also provide means for embolic protection during such an endovascular aortic valve implantation procedure.

In one aspect, there is described a system for transcatheter aortic valve treatment, comprising: an arterial access sheath adapted to be introduced into an access site at the left or right common carotid artery or left or right subclavian artery, wherein the arterial access sheath has a first lumen sized and shaped to receive a valve delivery system configured to deliver a prosthetic valve into a heart or aorta through the arterial access sheath, the first lumen having a first opening at a proximal region of the arterial access sheath and a distal opening at a distal region of the arterial access sheath, first lumen is sized to fit therethrough the valve delivery system; the valve delivery system, wherein the valve delivery system fits within the first lumen of the arterial access sheath and is configured to deliver a prosthetic aortic valve; an embolic protection element coupled to the arterial access sheath, the embolic protection element configured to be positioned in an aorta such that the embolic protection element causes blood flow to be redirected away from an orifice of an artery that branches off of the aorta; and at least one capture filter coupled to the arterial access sheath, the at least one capture filter configured to be positioned in the aorta in a position relative to the embolic protection element.

In another aspect, there is described A method of providing embolic protection during an endovascular aortic valve implantation procedure, comprising: delivering an embolic protection element to an aorta via an access site at the left or right common carotid artery or left or right subclavian artery; positioning at least a portion of the embolic protection element in the aorta; causing the embolic protection element to redirect blood flow in the aorta away from an orifice of an artery that branches off the aorta; and positioning a capture filter in the aorta such that the capture filter captures embolic debris in the aorta.

Other aspects, features and advantages should be apparent from the following description of various embodiments, which illustrate, by way of example, the principles of the disclosure.

### BRIEF DESCRIPTION OF THE DRAWINGS

FIG. 1 shows a side view of an exemplary access sheath having an occlusion element mounted on the sheath.

FIG. 2 shows a side view of an exemplary access sheath having a filter element mounted on the sheath.

FIG. 3 shows a front view of the filter element.

FIGS. 4, 5A, and 5B show alternate embodiments of the access sheath.

FIG. 6 schematically depicts a view of the vasculature showing normal circulation.

FIGS. 7A and 7B shows other embodiments of an access sheath deployed in the vasculature.

FIGS. 8A and 8B shows other embodiments of an access sheath deployed in the vasculature.

FIG. 9 shows another embodiment of an access sheath deployed in the vasculature.

FIG. 10 shows another embodiment of an access sheath deployed in the vasculature.

FIG. 11 shows another embodiment of an access sheath deployed in the vasculature with an occlusion element occluding the left common carotid artery.

FIG. 12 shows another embodiment of an access sheath deployed in the vasculature with an occlusion element occluding the right common carotid artery.

FIG. 13 shows another embodiment of an access sheath deployed in the vasculature with an occlusion element occluding the innominate artery.

FIG. 14 shows a delivery system deploying an endovascular prosthetic valve via an access sheath 110 and guidewire 119.

FIGS. 15A-15B show embodiments wherein a filter is sized and shaped to be deployed across the ostium of the artery.

FIG. 15C shows an alternate embodiment wherein the filter is sized and shaped to be deployed in the artery distal to the ostium of the artery.

FIGS. 16A, 16B, and 16C show embodiments wherein the filter is delivered through the access sheath.

FIGS. 17A, 17B, 17C, and 17D show embodiments of an access sheath with a filter, wherein the filter is sized and shaped to be deployed in the aortic arch across the ostia of all the head and neck vessels or across the aortic arch.

FIGS. 18A and 18B show alternate embodiments of an access sheath with a filter, wherein the filter is sized and shaped to be deployed in the ascending aorta.

FIG. 19 shows an embodiment of an access sheath with an occlusion element, wherein the occlusion element is sized and shaped to be deployed in the ascending aorta.

FIGS. 20A and 20B show alternate embodiments for delivering a prosthetic valve.

FIG. 21 shows another embodiment for delivering a prosthetic valve.

FIG. 22 shows an embodiment of a transcarotid prosthetic aortic valve and delivery system.

FIG. 23 shows an alternate embodiment of a transcarotid prosthetic aortic valve and delivery system.

FIG. 24 shows an embodiment of a transcarotid prosthetic aortic valve and delivery system with a filter element.

FIG. 25 shows an embodiment of a transcarotid prosthetic aortic valve and delivery system with an occlusion element.

FIGS. 26A-26G show an alternative embodiment of a filter configuration.

FIGS. 27A and 27B show an alternative embodiment of a filter configuration.

FIGS. 28A and 28B show an alternative embodiment of a filter configuration.

FIGS. 29A, 29B, 29C, and 29D show an alternative embodiment of a filter configuration.

FIG. 30 shows an embodiment of an aortic filter system.

FIGS. 31A, 31B, and 31C show an alternative embodiment of an aortic filter system.

FIG. 32 shows an alternative embodiment of an access sheath.

FIG. 33 shows another alternative embodiment of an access sheath with a filter.

FIGS. 34A and 34B show an embodiment of an aortic filter.

FIGS. 35A and 35B show an embodiment of an aortic filter and support wire system.

FIG. 36 shows an alternative embodiment of an aortic filter and support wire.

FIGS. 37A and 37B show an alternative embodiment of an access sheath with a filter.

FIG. 38 shows an alternative embodiment of an access sheath with a filter.

FIG. 39 shows an embodiment of an access sheath with multiple filters.

FIG. 40 shows an embodiment of an access sheath with multiple filters.

FIG. 41 shows an alternative embodiment of an access sheath with multiple filters.

FIG. 42 shows an alternate embodiment of an access sheath and filter.

FIG. 43 shows an additional alternate embodiment of an access sheath and filter.

FIG. 44 shows an alternate embodiment of an access sheath and filter.

FIGS. 45A and 45B show an alternate embodiment of an access sheath and filter.

FIG. 46 shows an alternate embodiment of an access sheath and filter.

FIG. 47 shows an alternate embodiment of an access sheath and filter.

FIG. 48 shows an alternate embodiment of an access sheath and aortic filter.

FIG. 49 shows an alternate embodiment of an access sheath, aortic filter, and guidewire assembly.

FIGS. 50A, 50B, and 50C illustrate an embodiment of an aortic filter being retracted into an access sheath via a guidewire.

FIGS. 51A, 51B, and 51C show an alternate embodiment of an aortic filter with support wire.

FIGS. 52-58 show alternative embodiments.

### DETAILED DESCRIPTION

Disclosed herein are devices and methods that allow arterial access, such as transcarotid access via the (left or right) common carotid artery, or subclavian access via the subclavian artery to the native aortic valve, and implantation of a prosthetic aortic valve into the heart or aorta. The devices and methods also provide means for embolic protection during such an endovascular aortic valve implantation procedure.

In an embodiment, transcarotid or subclavian access to the aortic valve is accomplished via either a percutaneous puncture or direct cut-down to the artery. A cut-down may be advantageous due to the difficulty of percutaneous vessel closure of larger arteriotomies in the common carotid artery. If desired, a pre-stitch may be placed at the arteriotomy site to facilitate closure at the conclusion of the procedure. An access sheath with associated dilator and guidewire is provided which is sized to fit into the common carotid or subclavian artery. The access sheath is inserted into the artery inferiorly towards the aortic arch. Either the left or the right common carotid or subclavian artery may be selected as the access site, based on factors including, for example, the disease state of the proximal artery and/or the aorta and the angle of entry of the carotid or innominate artery into the aorta. The carotid artery may then be occluded distal to the access site. If the access is via a direct surgical cut-down and arteriotomy, the occlusion may be accomplished via a vascular clamp, vessel loop, or Rummel tourniquet. Alternately, the access sheath itself may include an occlusion element adapted to occlude the artery, for example an occlusion balloon, to prevent embolic particulates from entering the carotid artery distal to the access site during the procedure.

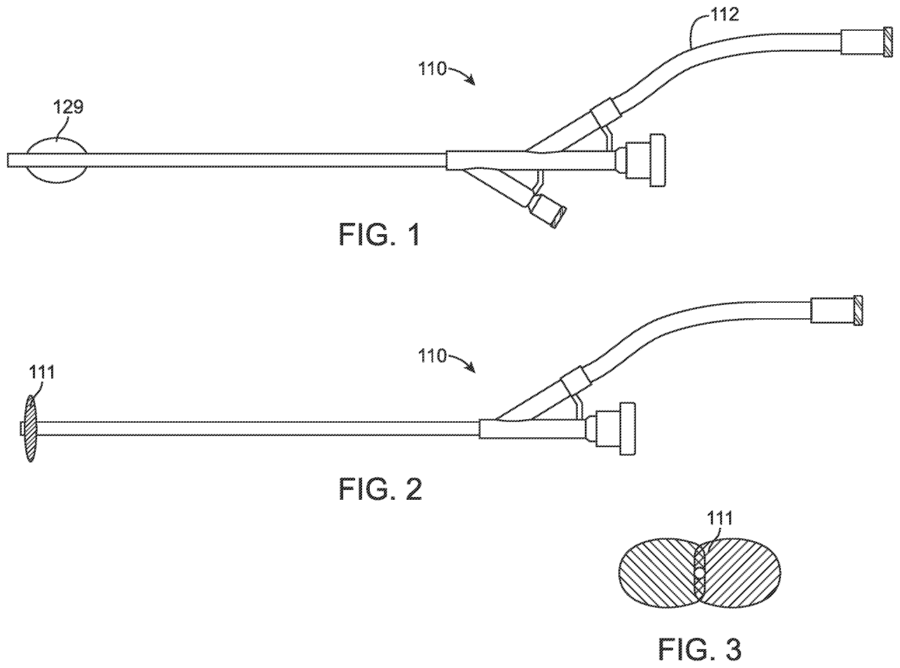

FIG. 1 shows a side view of an exemplary arterial access sheath 110 (or arterial sheath 110) formed of an elongate body having an internal lumen. In an embodiment, the sheath has a working length of 10-60 cm wherein the working length is the portion of the sheath that is insertable into the artery during use. The lumen of the sheath has an inner diameter large enough to accommodate insertion of an endovascular valve delivery system, such as an 18 French to 22 French (0.236″ to 0.288″) system. In an embodiment, the delivery system has an inner diameter as low as about 0.182″. The access sheath 110 can have an expandable occlusion element 129 positioned on the access sheath. The occlusion element 129 is configured to be expanded to a size for occluding flow through the artery. The occlusion element 129 may be placed anywhere in the artery or aorta. In an embodiment, the occlusion element is an occlusion balloon.

Once the sheath 110 is positioned in the artery, the occlusion element 129 is expanded within the artery to occlude the artery and possibly anchor the sheath into position. The arterial access sheath 110 may include a Y-arm for delivery of contrast or saline flush, for aspiration, and/or may be fluidly connected to a shunt, wherein the shunt provides a shunt lumen or pathway for blood to flow from the arterial access sheath 110 to a return site such as a venous return site or a collection reservoir. In this regard, a retrograde or reverse blood flow state may be established in at least a portion of the artery. The sheath 110 may also include a Y-arm for inflation of the occlusion balloon via an inflation lumen, and a hemostasis valve for introduction of an endovascular valve delivery system into the sheath. Alternately, the sheath 110 may include an actuating element if the occlusion element is a mechanical occlusion structure. The endovascular valve delivery system may include a prosthetic valve and a delivery catheter. In an embodiment, the delivery catheter has a working length of 30, 40, 60, 70, or 80 cm.

In an embodiment, aspiration may be applied to the artery via the access sheath 110. In this regard, the access sheath 110 can be connected via a Y-arm 112 to an aspiration source, so that embolic debris may be captured which may otherwise enter the remaining head and neck vessels, or travel downstream to lodge into peripheral vessels. The aspiration source may be active, for example a cardiotomy suction source, a pump, or a syringe. Alternately, a passive flow condition may be established, for example, by fluidly connecting the Y-arm 112 to a shunt, which in turn is connected to a lower-pressure source such as a collection reservoir at atmospheric or negative pressure, or a venous return site in the patient. The passive flow rate may be regulated, for example, by controlling the restriction of the flow path in the shunt.

In an embodiment, the access system may be equipped with one or more embolic protection elements to provide embolic protection for one or both carotid arteries. For example, a filter may be included in the access system to provide embolic protection for one or both carotid arteries. In a variation of this embodiment, the filter is deployed via the contralateral carotid, brachial or subclavian artery, and positioned in the aortic arch across the ostium. If the sheath access site is the left common carotid artery, the filter may be positioned across the ostium of the innominate (also known as brachiocephalic) artery. If the sheath access site is the right common carotid artery, the filter may be positioned across the ostium of the left common carotid artery. In a variation of this embodiment, the filter is deployed across both the innominate and left common carotid artery, or across all three head and neck vessels (innominate artery, left common carotid artery, and left subclavian artery). The filter element may be built-in to the access sheath 110. In an embodiment, the filter element may be a separate element which is compatible with the access sheath 110. For example, the filter element may be a coaxial element which is slideably connected to the access sheath or an element which is placed side-by-side with the access sheath. The filter element may comprise an expandable frame, so that it may be inserted into the artery in a collapsed state, but then expanded at the target site to position the filter element across the opening of the artery or arteries.

FIG. 2 shows a side view of an exemplary access sheath 110 having a filter element 111 mounted on the sheath. FIG. 3 shows a front view of the filter element 111 showing an exemplary profile of the filter element 111. In the embodiment of FIG. 3, the filter element 111 is sized and shaped to fit within and block the head and neck vessels. In an embodiment, the deployed filter has a long dimension of about 2, 3, 4, or 5 cm and a short dimension of about 1, 1.5, or 2 cm. The profile shown in FIG. 3 is for example and it should be appreciated that the shape of the filter element 111 may vary. For example, the shape of the filter element may be oval, round, elliptical, or rectangular. The filter material may be woven or knitted textile material, or may be a perforated polymer membrane such as polyurethethane. The filter porosity may be 40, 100, 150, 200, or 300 microns, or any porosity in between. The expandable frame of the filter element may be made from spring material such as stainless steel or nitinol wire or ribbon.

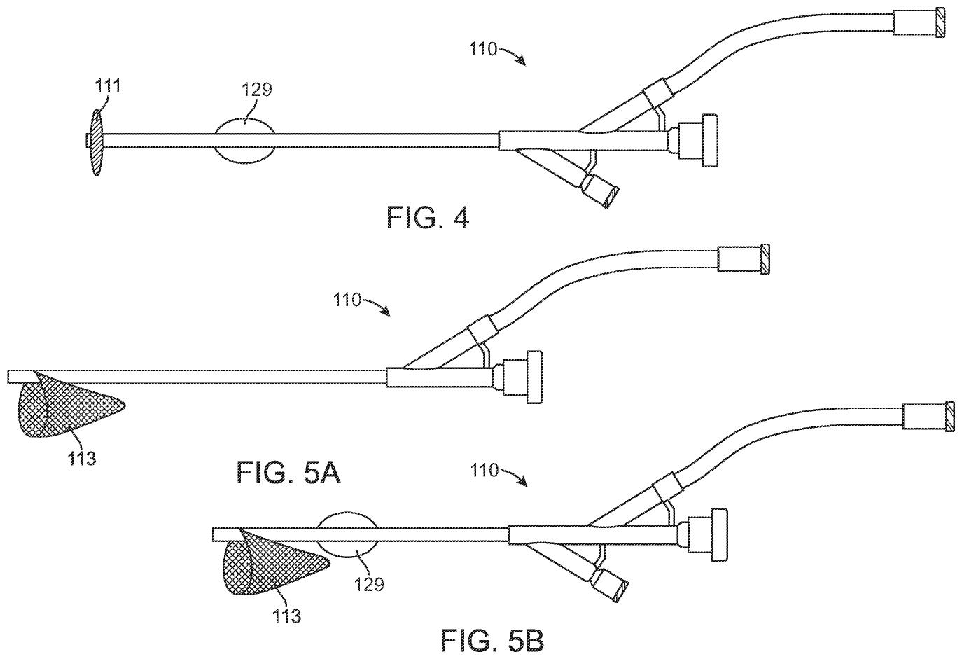

In the embodiment with the filter element, occlusion and/or aspiration means may still be part of the system, to provide embolic protection during filter deployment before the valve implantation and filter retrieval after valve implantation. The filter element itself may be a primary method of embolic protection during the implantation procedure. The sheath 110 may also be equipped with both an occlusion element 129 and a filter element 111, as shown in FIG. 4.

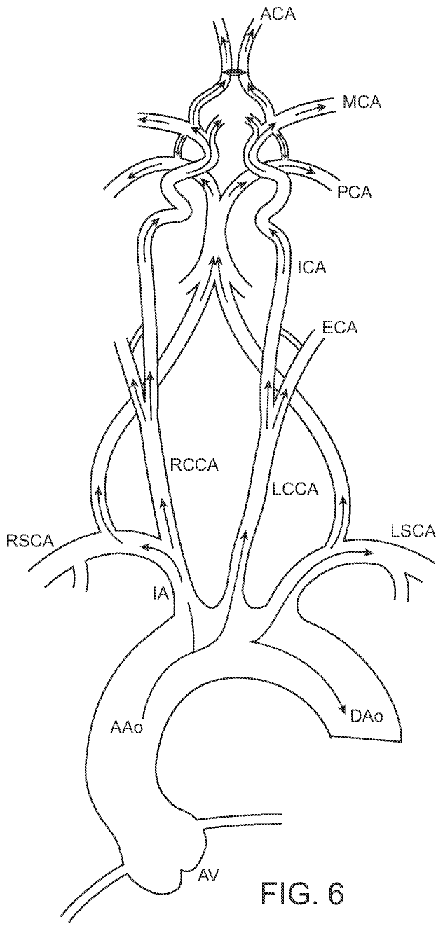

In another variation of this embodiment, shown in FIG. 5A, the sheath 110 includes an aortic filter element 113 which is sized and shaped to be deployed across the ascending aorta and thus protect all the head and neck vessels from embolic debris. The shape of the filter element may vary. In an embodiment, the shape of the filter element may be a cone or a closed-end tube. The expandable frame of the filter frame is sized and shaped to traverse the entire diameter of the aorta when deployed. For example the expandable frame may be a loop which can expand from 12 to 30 mm in diameter. Alternately, the expandable frame may be a series of struts connected at one or both ends and which expand outwardly to deploy the filter element across the diameter of the aorta. The filter material may be woven or knitted textile material, or may be a perforated polymer membrane such as polyurethethane. The filter porosity may be about 40, 100, 150, 200, or 300 microns, or any porosity in between.

The expandable frame of the filter element may be made from spring material such as stainless steel or nitinol wire or ribbon. As with the previous variation, occlusion and aspiration means may be included in this variation to provide protection during filter deployment and filter retrieval. The aortic filter element 113 may be integral to the sheath, or be a separate device which is compatible with the sheath, for example may be coaxial or side-by-side with the access sheath. As shown in FIG. 5B, an embodiment of the sheath 110 may include both an aortic filter element 113 and an occlusion element 129.

FIG. 6 schematically depicts a view of the vasculature showing normal antegrade circulation. The blood vessels are labeled as follows in FIG. 6: ACA: anterior cerebral artery; MCA: middle cerebral artery; PCA: posterior cerebral artery; ICA: internal carotid artery; ECA: external carotid artery; LCCA: left common carotid artery; RCCA: right common carotid artery; LSCA: left subclavian artery; RSCA: right subclavian artery; IA: innominate artery; AAo: Ascending aorta; DAo: descending aorta; AV: aortic valve.

In certain situations, it may be desirable to provide a mechanism for perfusing the carotid artery upstream of the entry point of the access sheath 110 into the carotid or innominate artery. If the access sheath 110 is similar in size to the carotid or innominate artery, flow through the artery may be essentially blocked by the access sheath when the sheath is inserted into the artery. In this situation, the upstream cerebral vessels may not be adequately perfused due to blockage of the carotid artery by the sheath. In an embodiment of the access sheath 110, the sheath includes a mechanism to perfuse the upstream carotid and cerebral vessels.

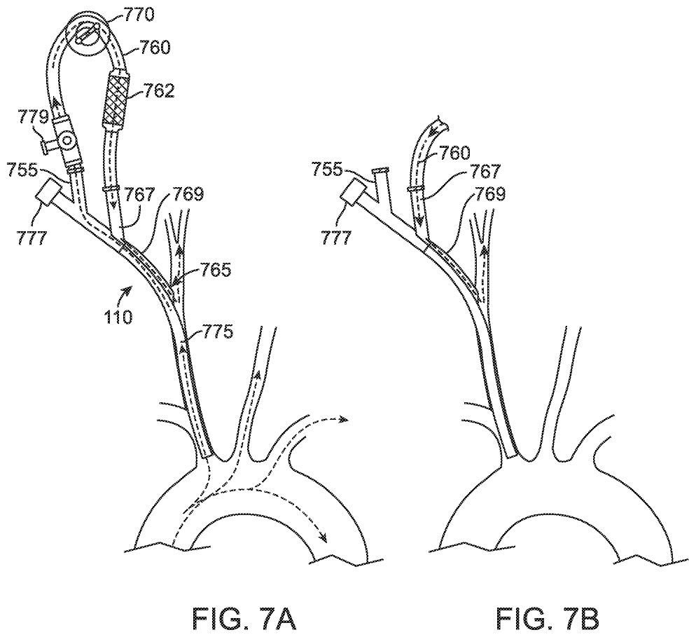

FIG. 7A shows an exemplary embodiment of such an access sheath 110 deployed in the vasculature. A proximal portion of the access sheath has two parallel, internal lumens that are part of a single monolithic structure of the access sheath. A first lumen 775 extends from the proximal end of the sheath to the distal tip of the sheath and is fluidly connected on the proximal end to a shunt Y-arm 755 and a hemostasis valve 777 located at the proximal end of the sheath. The first lumen 775 is sized and shaped to receive and enable delivery of a transcatheter aortic valve and delivery system via the hemostasis valve 777. For example, the first lumen has a length such that its distal opening is positioned at the heart or aorta. A second lumen 769 is positioned adjacent the first lumen and extends from the proximal end of the sheath to a distal opening at a location mid-shaft 765 and is fluidly connected on the proximal end to a second, perfusion Y-arm 767. There is an opening on the distal end of the second lumen at the location 765. The second lumen 769 is sized and shaped to enable shunting of blood to the carotid artery distal of the access sheath insertion site. A radiopaque shaft marker may be positioned on the sheath at this location to facilitate visualization of this opening to the user under fluoroscopy. The perfusion lumen has a length such that the distal opening of the perfusion lumen can be positioned in and perfuse a distal carotid artery when in use. The proximal end of the first lumen has a proximal connector with the hemostasis valve 777 and a Y-arm. As mentioned, the hemostasis valve is sized to fit therethrough an arterial valve delivery system. The proximal end of the perfusion lumen also has a proximal connector. The proximal connectors and/or Y-arms permit a shunt to be attached.

The Y-arm 755 is removably connected to a flow shunt 760 which in turn is removably connected to the second Y-arm 767. The shunt defines an internal shunt lumen that fluidly connects the first lumen 775 to the second lumen 769. A stopcock 779 may be positioned between the Y-arm 755 and the flow shunt 760 to allow flushing and contrast injection while the shunt 760 is connected. When the sheath is positioned in the artery, arterial pressure drives blood flow into the distal end of the first lumen 775 of the arterial access sheath, out the first lumen from Y-arm 755, then into the shunt 760, and back into the sheath via the Y-arm 767. The blood then flows into the parallel lumen 769 and into the distal carotid artery at the location 765 to perfuse the vasculature distal of the arterial sheath 110. An in-line filter element 762 may be included in the flow shunt 760 so that emboli generated during the procedure are not perfused into the cerebral artery. In the event the sheath 110, shunt 760, and lumen 769 create a flow restriction that limits adequate perfusion, the flow shunt 760 may incorporate an active pump 770 to drive blood flow and provide the required level of cerebral perfusion. This may be especially true when the valve is being delivered through the first lumen 775 of the access sheath 110.

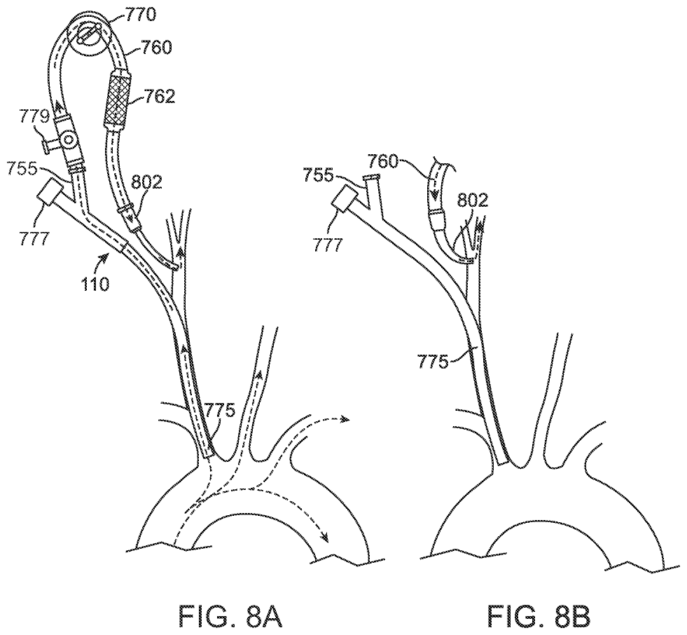

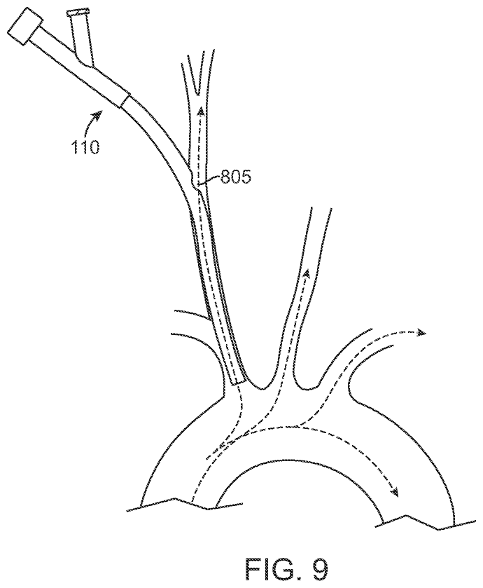

FIG. 7B shows a variation of the embodiment of FIG. 7A. The Y-arm 767 fluidly connects the shunt 760 to the parallel lumen 769 that re-introduces blood from the shunt 760 into the artery at location 765 when positioned in the artery. The shunt 760 in this embodiment is not fluidly connected to the first lumen 775 in the sheath. The shunt 760 rather than receiving blood from the access sheath via Y-arm 755 may be connected to another arterial blood source via a second sheath, for example a femoral or subclavian artery or the contralateral carotid artery. In this variation, the shunted blood flow is not restricted by the delivery of the valve through the first lumen 775 of the access sheath. In this embodiment, there is no need for a filter 762 in the shunt line, as the blood source is far from the treatment area and there is minimal risk of distal emboli in the shunted blood. The Y-arm 755 may still be used for flushing and contrast injection into the sheath. In another variation, shown in FIG. 8A, the arterial access sheath 110 has a single lumen 775 which is fluidly connected to a Y-arm 755 at the proximal region. The lumen 775 is sized and shaped to receive and enable delivery of a transcatheter aortic valve and delivery system via a hemostasis valve 777. The Y-arm 755 is connected to a flow shunt 760 which in turn is connected to a second arterial sheath 802 which is sized and shaped to be introduced into the carotid artery distal to arterial access point where the access sheath 110 is introduced. A stopcock 779 may be positioned between the Y-arm 755 and the flow shunt 760 to allow flushing and contrast injection while the shunt 760 is connected. When the sheath is properly positioned in the artery, the arterial pressure drives flow into the lumen 775 of the sheath 110, out the first lumen via Y-arm 755, then through the shunt 760, through the second catheter 802, and back into the carotid artery upstream from the arterial access point to perfuse the vasculature distal of the arterial sheath 110. As above, a filter element 762 may be included in the flow shunt 760 so that emboli generated during the procedure are not perfused into the cerebral artery. In the event the sheath 110 and shunt 760 experience flow restriction that limits adequate perfusion, for example when the valve is being delivered through the lumen 775 of the access sheath 110, the flow shunt may incorporate an active pump 770 to drive blood flow and provide the required level of cerebral perfusion.

FIG. 8B shows a variation of the embodiment of FIG. 8A. Here, the second arterial sheath 802 is removably or fixedly connected to a shunt or flow line 760 which in turn is connected to another arterial source via another sheath, for example a femoral or subclavian artery or the contralateral carotid artery. In this variation, the shunted blood flow is not restricted by the delivery of the valve through the lumen 775 of the access sheath. In this embodiment, there is no need for a filter 762 in the shunt line, as the blood source is far from the treatment area and there is minimal risk of distal emboli in the shunted blood. The Y-arm 755 may still be used for flushing and contrast injection into the sheath.

In the embodiments described above in FIGS. 7A and 7B and FIGS. 8A and 8B, the arterial access sheath 110 may include an occlusion element (not shown) at the distal end of the sheath, configured to occlude the carotid artery and to assist in prevention of emboli from entering the carotid artery. Additionally, in these embodiments, the flow shunt 760, and if applicable pump 770 and/or the second sheath 820 may be provided as separate components in a single kit to enable transcarotid access and carotid shunting during a catheter-based aortic valve replacement procedure.

Although the figures show sheath insertion in the common carotid artery, a similar sheath or sheath/shunt systems may be designed for sub-clavian access.

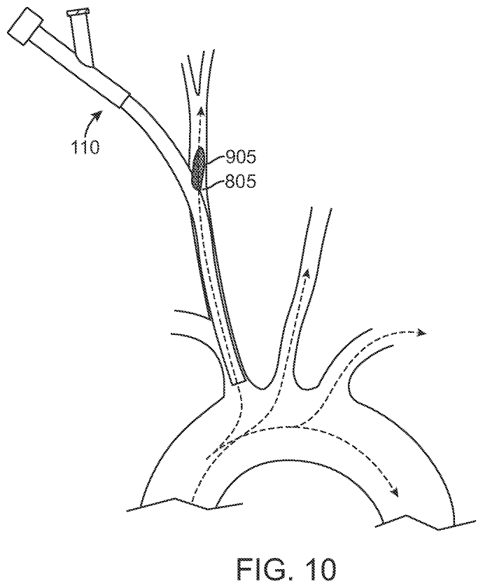

In another embodiment, the access sheath 110 may have at least one side opening 805 located between the distal end and the proximal end of the sheath 110, as shown in FIG. 9. A dilator may be positioned inside the sheath 110 to block the side opening 805 during insertion of the sheath. The dilator is used to aid in sheath insertion into the artery. When the access sheath 110 is inserted into the artery and the dilator is removed, the dilator no longer blocks the opening 805 so that blood may flow out of the access sheath 110 through the side opening 805 into the distal carotid artery. During introduction of the endovascular valve delivery system through the access sheath 110 and into the artery, the delivery system may restrict the flow through the sheath and artery and may reduce the level of cerebral perfusion. However, this period of the procedure is transient, and reduction of cerebral perfusion during this limited period of time should not present a clinical issue. In a variation of this embodiment as shown in FIG. 10, the side opening 805 may have a filter 905 that covers the opening 805. The filter 905 is configured to capture embolic debris so that the debris does not pass downstream towards the cerebral arteries. The filter 905 may be sized and shaped to bulge out of the sheath 110, so that when the endovascular valve is inserted into the sheath 110, the debris is not pushed forward and out the distal end of the sheath into the artery. In an embodiment, the filter is very thin, perforated film or woven material, similar in composition to embolic distal filter materials. Filter porosity may be about 150, 200, or 250 microns. Though FIGS. 9 and 10 show sheath insertion in the common carotid artery, a similar sheath may be designed for sub-clavian access, in which the sheath insertion site is farther from the carotid artery and the side opening may be placed correspondingly further towards the tip of the sheath.

The sheath in embodiments shown in FIGS. 7-10 may optionally include a positioning element which can be deployed once the sheath is inserted into the blood vessel. The positioning element can be used to position the sheath in the vessel such that the side opening 805 remains in a desired location inside the vessel. This positioning feature may take the form of a deployable protruding member such as a loop, braid, arm, or other protruding feature. This feature may be retracted during sheath insertion into the artery but deployed after the sheath is inserted and the opening is inside the vessel wall. Sheath retention may also be achieved, for example, by an eyelet or other feature in the Y-arm of the access sheath 110 which allows the sheath to be secured to the patient once positioned correctly.

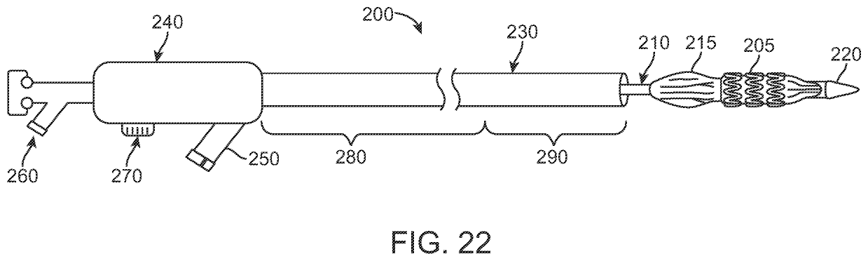

An exemplary valve and delivery system which has been configured to be delivered through the transcarotid access sheath 110 is shown in FIG. 22. The route from the transcarotid access site is fairly short and straight, as compared to the transfemoral or subclavian approach. As a result, the delivery system can be shorter and the proximal section can be quite rigid, both of which will allow greater push and torque control resulting in increased accuracy in positioning and deploying the prosthetic valve. The distal section has increased flexibility to allow accurate tracking around the ascending aorta and into position at the aortic annulus. Materials for the delivery system may include reinforced, higher durometer, and/or thicker walled materials as compared to current delivery systems to provide this increased rigidity.

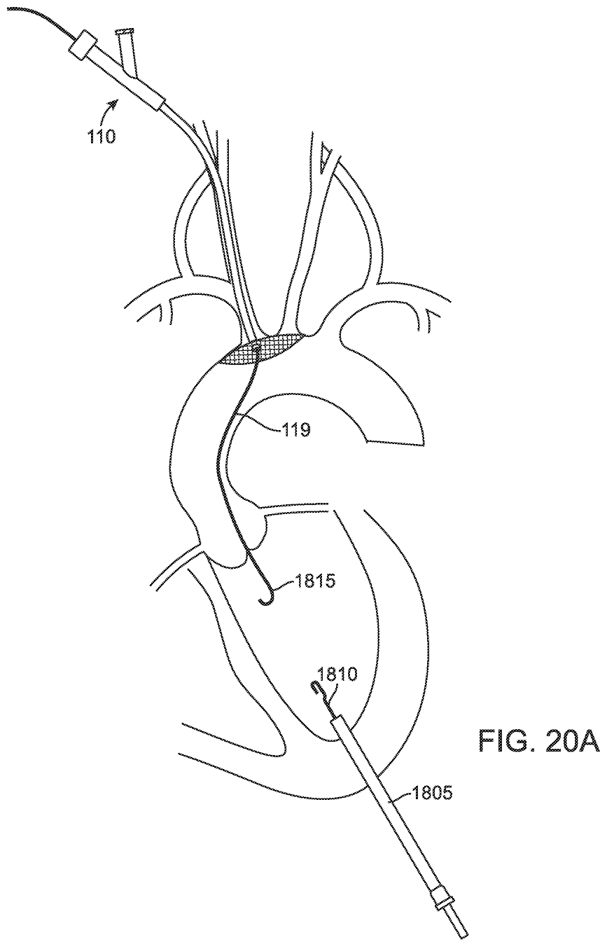

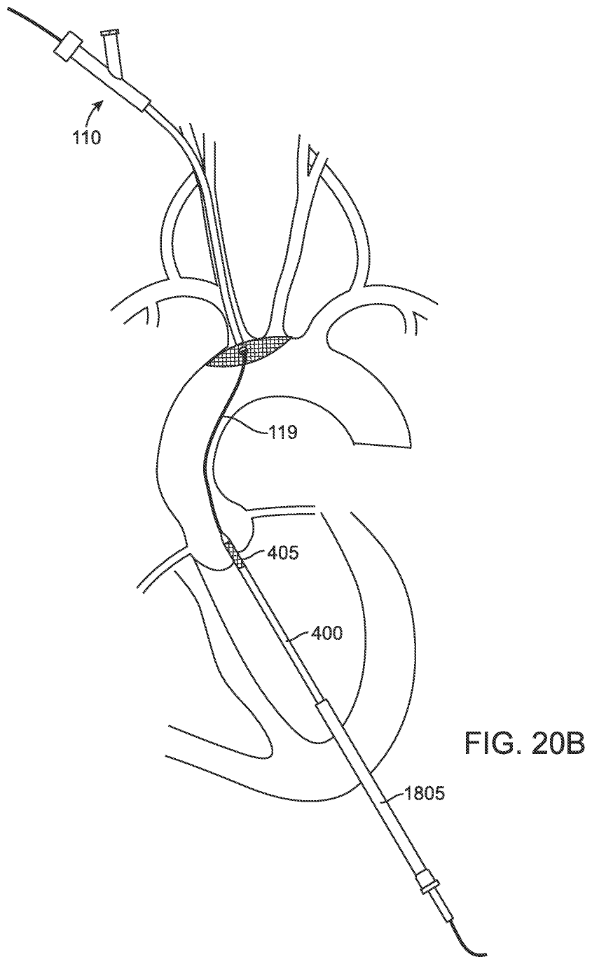

The balloon expandable prosthetic aortic valve 205 is mounted on the distal end of an endovascular valve delivery system 200. The delivery system has a distal tapered tip 220 and an expandable balloon 215 on the distal end of an inner shaft 210. In an embodiment, the system also has an outer sleeve, such as for example a pusher sleeve 230, that is slidable along the long axis of the device and which maintains the valve in position on the balloon during delivery. A proximal control assembly contains a mechanism for retracting the pusher sleeve, such as a sliding button 270 on a proximal handle 240. In FIG. 20, the pusher sleeve 230 is shown retracted from the valve and proximal balloon so that the valve can be expanded without interference from the pusher sleeve 230. A connector 250 allows connection of an inflation device to the balloon inflation lumen of the balloon 215. A proximal rotating hemostasis valve 260 allows system flushing as well as sealing around a guidewire (not shown) as the valve delivery system is being advanced over the guidewire and into position.

The working length of the valve delivery system is configured to allow delivery of the valve to the aortic annulus from a transcarotid access site. Specifically, the working length of the valve delivery system 200 can be between 45 and 60 cm. The delivery system shaft is also configured for delivery from a right or left carotid access site. Specifically, the shaft has a proximal stiff section 280 and a more flexible distal section 290. In an embodiment, the distal section is 2 to 4 times more flexible than the proximal stiff section. In an embodiment, the distal flexible section is between one quarter to one third the total working length of the valve delivery system. Specifically, the distal flexible section is in the range 10 cm to 20 cm. In an alternate embodiment, the valve delivery system has a transition section of one or more flexible lengths which fall between the flexibility of the distal flexible section and the proximal flexible section.

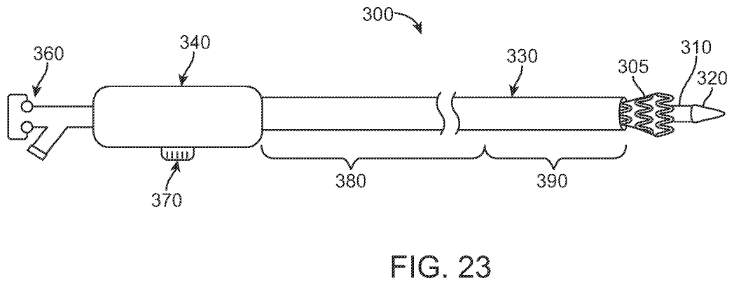

Another exemplary valve and delivery system configured for transcarotid delivery is shown in FIG. 23. The self-expanding prosthetic aortic valve 305 is mounted on the distal end of an endovascular valve delivery system 300. The delivery system has a distal tapered tip 320 on the distal end of an inner shaft 310. The valve 305 is positioned on the inner shaft 310 and contained in a retractable sleeve 330 that can slide along the longitudinal axis of the device. A proximal control assembly contains a mechanism for retracting the retractable sleeve, such as a sliding button 370. In an embodiment, the design of the valve 305 and sleeve 330 are such that the sleeve can be readvanced in a distal direction to abut and to collapse the valve so that the valve 305 can be re-positioned if the first position was inaccurate. A proximal rotating hemostasis valve 360 allows system flushing as well as sealing around a guidewire (not shown) as the valve delivery system is being advanced over the guidewire and into position.

As with the previous embodiment, the working length of the valve delivery system is configured to allow delivery of the valve to the aortic annulus from a transcarotid access site. Specifically, the working length of the valve delivery system 300 is between 45 and 60 cm. The delivery system shaft is also configured for delivery from a right or left carotid access site. Specifically, the shaft has a proximal stiff section 380 and a more flexible distal section 390. In an embodiment, the distal section is 2 to 4 times more flexible than the proximal stiff section. In an embodiment, the distal flexible section is between one quarter to one third the total working length of the valve delivery system. Specifically, the distal flexible section is in the range 10 cm to 20 cm. In an alternate embodiment, the valve delivery system has a transition section of one or more flexible lengths which fall between the flexibility of the distal flexible section and the proximal flexible section.

Exemplary methods of use are now described. In an embodiment, a general method includes the steps of forming a penetration from the neck or shoulder region of a patient into a wall of a common carotid artery; introducing an access sheath through the penetration with the tip directed inferiorly towards the ostium of the artery; inserting a guide wire through the access sheath into the ascending aorta and across the native aortic valve; and introducing a prosthetic valve through the access sheath and percutaneously deploying the prosthetic valve at or near the position of the native aortic valve. In an embodiment, the artery is occluded distal (upstream) from the tip of the sheath.

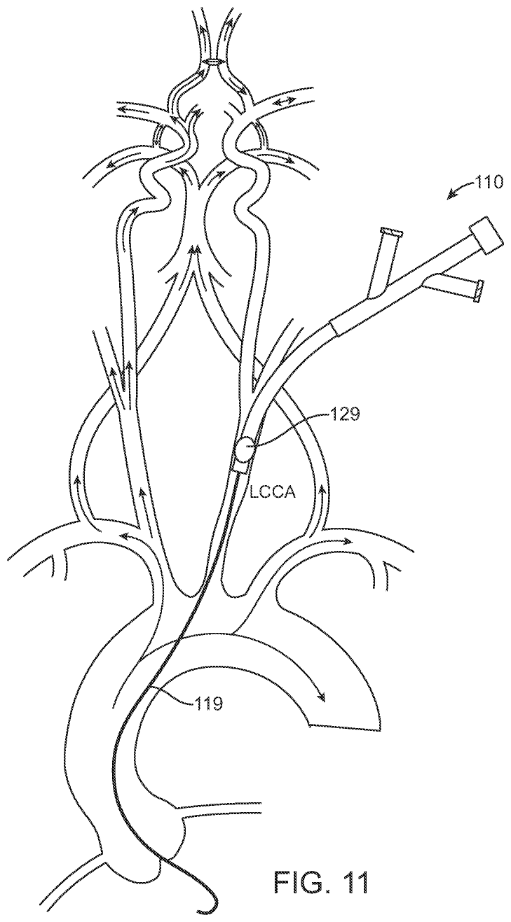

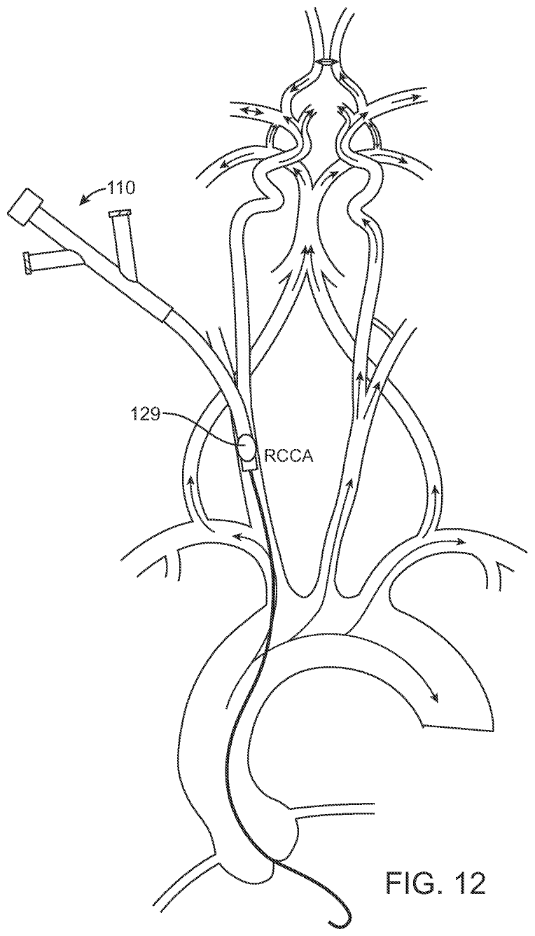

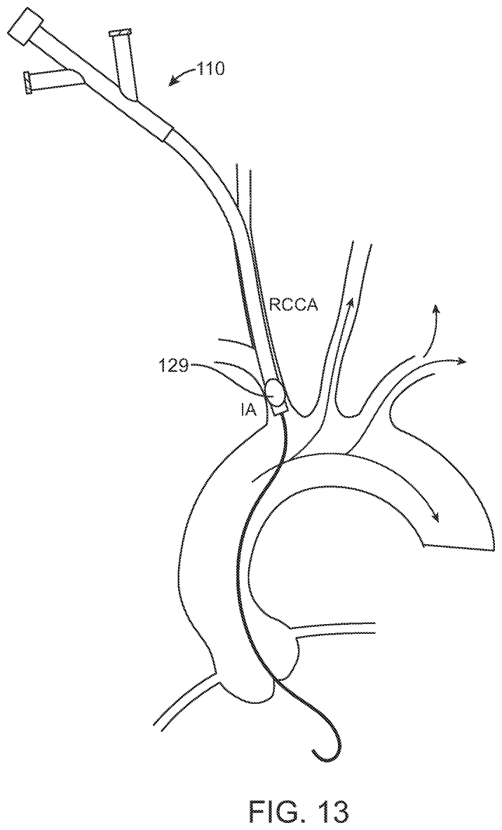

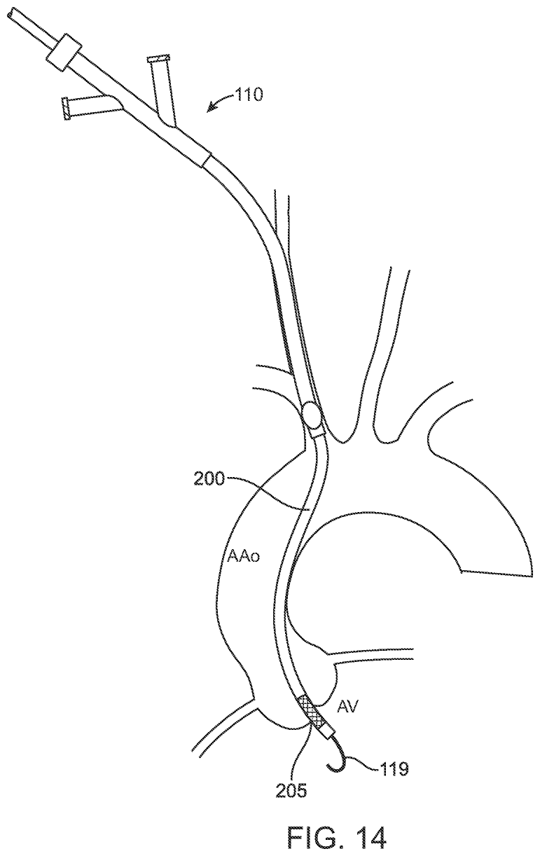

In particular, the access sheath 110 is first inserted into the vasculature such as via either a percutaneous puncture or direct surgical cut-down and puncture of the carotid artery. As mentioned, a transcarotid approach to the aortic valve may be achieved via the LCCA. Once properly positioned, the occlusion element 129 may be expanded to occlude the LCCA, as shown in FIG. 11. In another embodiment, a transcarotid approach to the aortic valve may be achieved via the RCCA, with the occlusion element 129 occluding the RCCA, as shown in FIG. 12. In another embodiment, a transcarotid approach to the aortic valve may be achieved via the RCCA, with the occlusion element 129 occluding the innominate artery IA, as shown in FIG. 13. The occlusion achieved via the occlusion element 129 can also be achieved via direct clamping of the carotid vessel, e.g. with a vascular clamp, vessel loop or Rummel tourniquet.

Once the access sheath is positioned, an embolic protection system or means can be deployed to the aorta or other site the lumen of the access sheath. The embolic protection means is deployed via occlusion, aspiration, and/or filter elements, access to the aortic valve is obtained via a guidewire 119 (such as a 0.035″ or 0.038″ guidewire) inserted into the sheath 110 and directed inferiorly into the ascending aorta and across the native aortic valve. Pre-dilation of the native aortic valve can be performed with an appropriately sized dilation balloon, for example a valvuloplasty balloon, before valve implantation. The guidewire 119 is used to position a balloon across the valve and the balloon is inflated, deflated, and then removed while the guidewire remains in place.

An endovascular prosthetic valve 205 and delivery system 200 is then inserted through the access sheath 110 over the guidewire 119 and the valve 205 positioned at the site of the native aortic valve. The prosthetic valve 205 is then implanted. At the conclusion of the implantation step, the implanted prosthetic valve 205 function can be accessed via ultrasound, contrast injection under fluoroscopy, or other imaging means. Depending on the design of the delivery system 200, the prosthetic valve 205 may be adjusted as needed to achieve optimal valve function and position before final deployment. The delivery system 200 and guidewire 119 are then removed from the access sheath 110. After removal of the delivery system 200 and guidewire 119, the embolic protection elements are removed. Aspiration may continue during this time to capture any embolic debris caught in the sheath tip, occlusion element and/or filter elements.

The access sheath 110 is then removed and the access site is closed. If the access was a surgical cutdown direct puncture, the vessel is closed either via tying off the pre-placed stitch or with manual suturing or with a surgical vascular closure device, as described in more detail below. If the access was percutaneous, percutaneous closure methods and devices may be employed to achieve hemostasis at the access site. In an embodiment, the closure device is applied at the site of the penetration before introducing the arterial access sheath through the penetration. The type of closure device can vary.

The access site described above is either the left or right common carotid artery. Other access sites are also possible, for example the left or right subclavian artery or left or right brachial artery. These arteries may require longer and/or more tortuous pathways to the aortic valve but may offer other advantages over a carotid artery access, for example the ability to work away from the patient's head, the ability to avoid hostile neck anatomy such as previous carotid endarterectomy or other cervical surgery or radiation, or less risky in case of access site complication. In addition, carotid artery disease, or small carotid arteries may preclude common carotid artery access. In the case of any of these access sites, occlusion, aspiration, and/or filtering the head and neck vessels during TAVI may increase the speed and accuracy of the procedure, and decrease the rate of embolic complications.

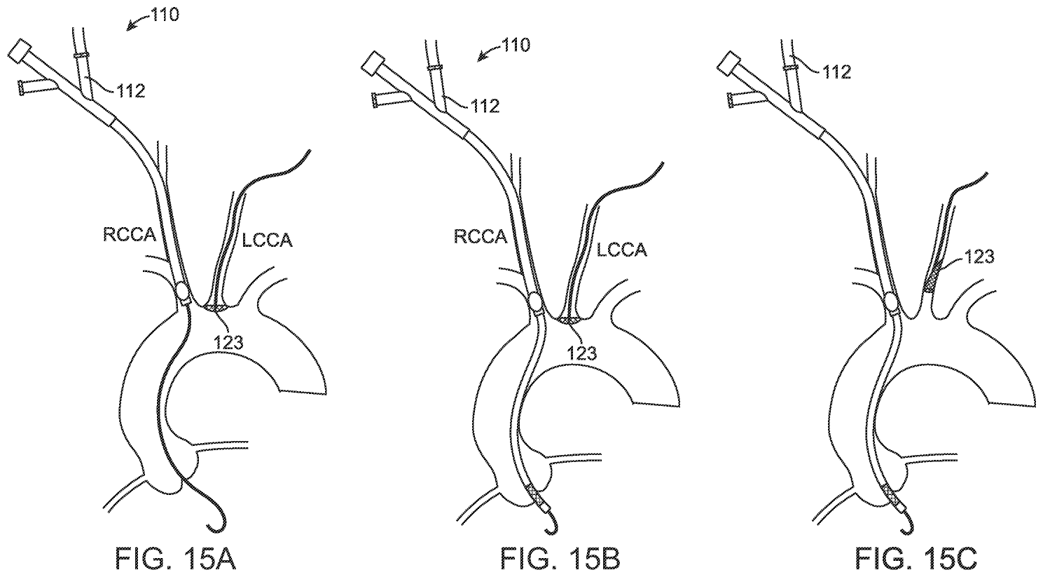

Various systems of embolic protection were described above including occlusion elements and filters. Additional embodiments that incorporate filters as means of embolic protection for all the head and neck vessels are now described. FIGS. 15A and 15B show embodiments wherein a filter 123 is sized and shaped to be deployed across the ostium of the artery contralateral (on the opposite side) to the carotid artery being accessed (left carotid artery if the right carotid artery is accessed, or innominate artery if the left carotid artery is accessed.) FIG. 15C shows an alternate embodiment, wherein the filter 123 is sized and shaped to be deployed in the artery distal to the ostium of the artery. The filter 123 can be placed via the contralateral carotid access site, or via a brachial or subclavian artery access site. Blood flow may proceed antegrade through the filter into the contralateral artery, while preventing the flow of embolic particles to the head and neck circulation.

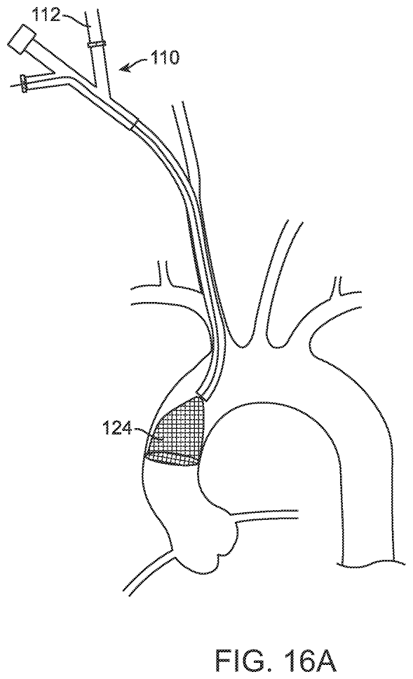

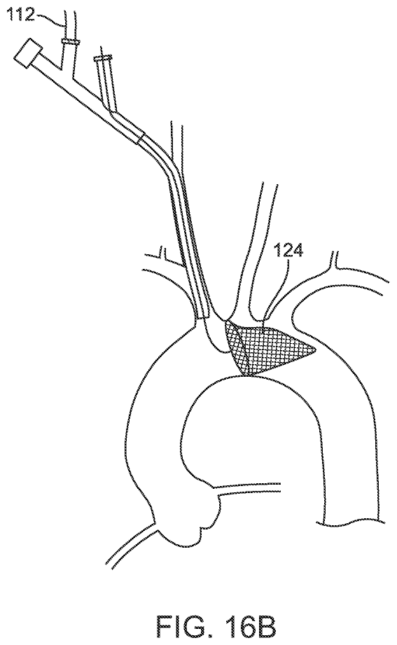

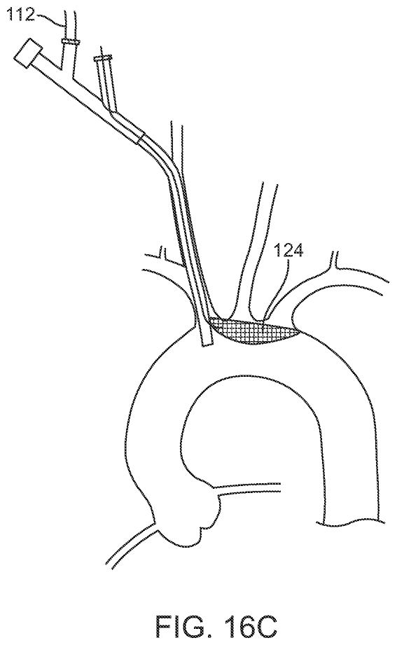

FIGS. 16A, 16B, and 16C show alternate embodiments wherein a filter is sized and shaped to be delivered through a lumen of the access sheath 110, either the main lumen or a separate lumen. In FIG. 16A, the filter 124 is sized and shaped to be deployed in the ascending aorta. In this embodiment, the valve is delivered through the filter material and into the aortic valve position. The filter may have a pre-formed slit or opening to allow the passage of the valve through the filter. The pre-formed slit(s) can be formed on the filter material to allow the passage of the valve though the filter while minimizing the size of the hole(s) created from the slit. For example the pattern of the slit(s) to create a one-way valve where the valve system can pass towards the heart but the slit(s) will be closed by the blood flowing away from the heart. Alternately, the material may be punctured, for example by an introducer needle, which can then deliver the guidewire through the filter material and across the native valve. The valve delivery system is then advanced over the guidewire through the filter material and to the target site.

In FIG. 16B, the filter 124 is sized and shaped to be delivered in the aortic arch such that it contacts the walls of the arch. In FIG. 16C, the filter 124 is sized and shaped to be delivered across the opening of both the head and neck vessels at the same time. In this embodiment, the filter may exit the access sheath through a side port to aid in positioning the filter in the superior aspect of the aortic arch. In the embodiments shown in FIGS. 16B and 16C, the filter 124 is downstream of the sheath opening and the valve does not have to traverse the filter to be delivered. The embodiments in FIGS. 16B and 16C may be delivered through the main sheath lumen and be positioned in the aorta through the help of the pulsing blood flow leaving the heart. As the filters in FIGS. 16B and 16C are delivered out of the sheath and into the aorta, the blood flow tugs or pulls on the filter material and naturally aides to position the filter distal of the sheath. The devices resides in that position until removed back through the sheath at the end of the procedure.

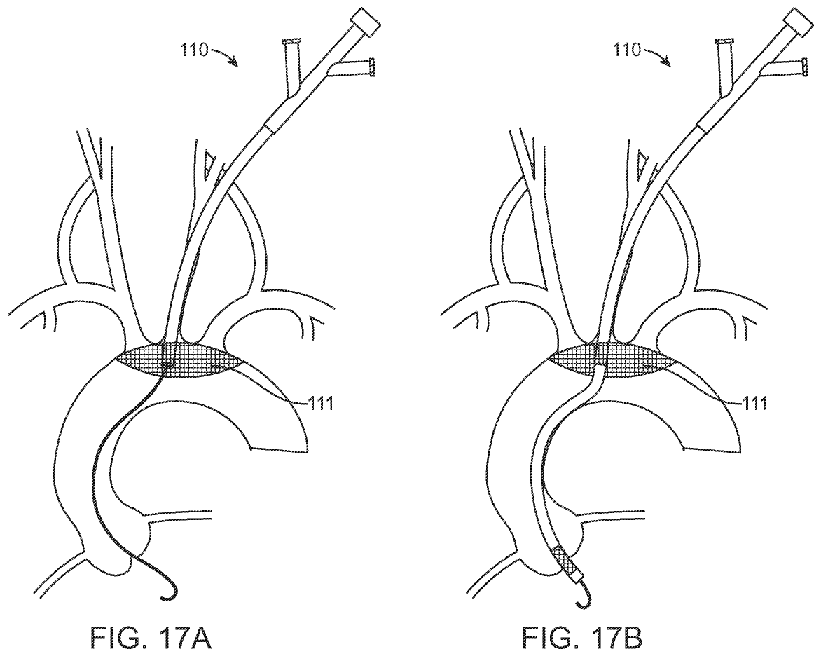

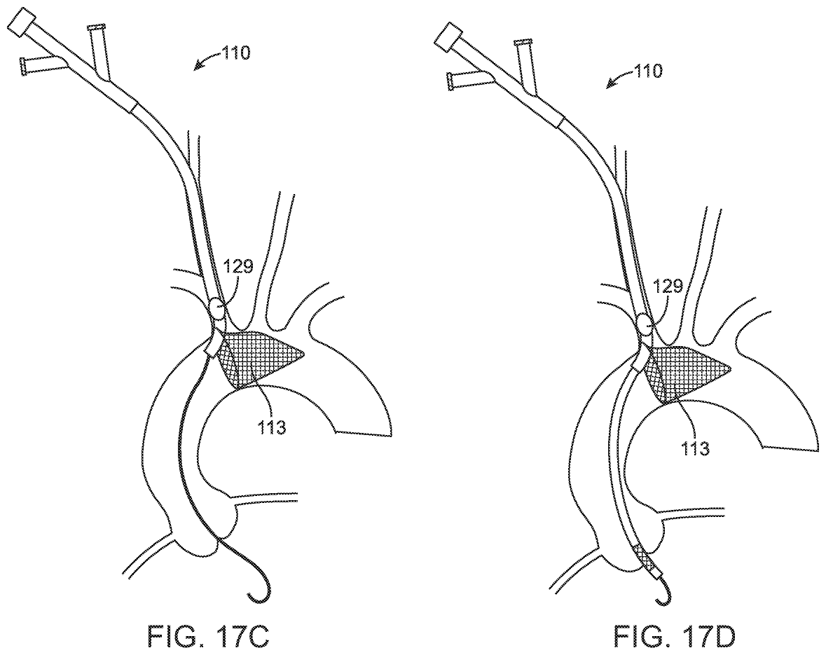

In another embodiment, shown in FIGS. 17A, 17B, 17C and 17D, an embolic filter is built onto or on the access sheath 110 and deployed in the aortic arch. In FIGS. 17A and 17B, the filter 111 is sized and shaped to be deployed across the superior aspect of the aortic arch, covering the ostia of some or all the head and neck vessels. In this embodiment, as both the access site carotid artery and the contralateral carotid artery are protected by the filter 111, an occlusion balloon is not required for embolic protection on the sheath during the valve implantation procedure. However, as there may be risk of embolic debris during deployment and retrieval of the filter, it may be desirable to retain the occlusion balloon and aspiration function as embolic protection during deployment of the filter prior to valve implantation, and retrieval of the filter after valve implantation. In FIGS. 17C and 17D, the aortic filter 113 is sized and shaped to be deployed across the aorta within the aortic arch, so that all downstream vessels are protected by the filter 113 from embolic debris during the valve implantation procedure.

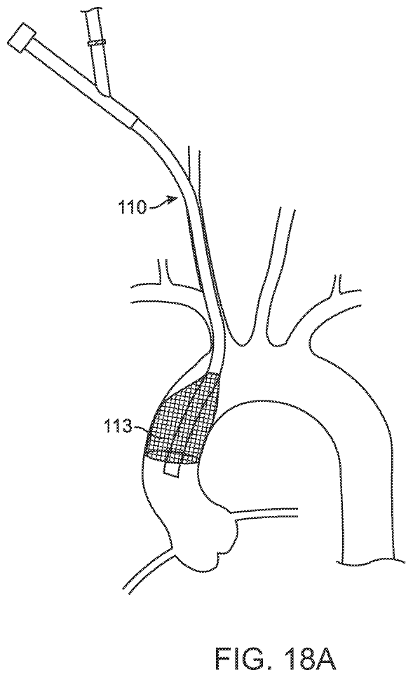

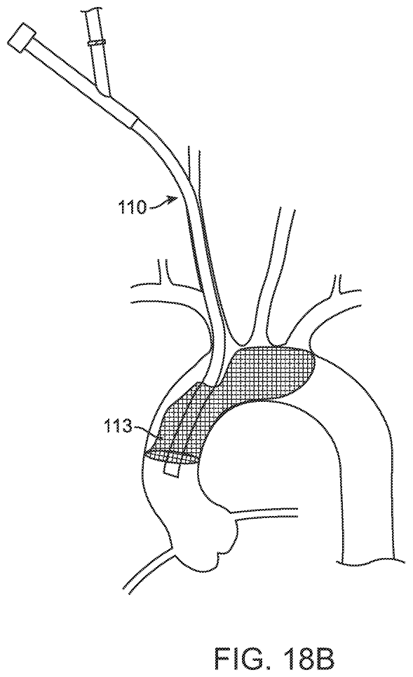

In another embodiment, shown in FIGS. 18A and 18B, the aortic filter is built onto or on the access sheath 110 and deployed in the ascending aorta. As shown in FIG. 18A, the aortic filter 113 is attached to the sheath 110 on the distal portion of the sheath. During use the distal portion of the access sheath is positioned in the ascending aorta, and then the filter 113 is expanded across the aorta so that all downstream vessels are protected by the filter. Deployment of the filter may be accomplished, for example, by a retractable sleeve on the outside of the sheath, which, when retracted, exposes the expandable filter. In one configuration, the filter deployed length may be varied depending on how much the retractable sleeve is pulled back. This control may allow the user to expand the filter to different sizes depending on the length and diameter the filter. Alternately, the filter may be pushed forward by a wire frame or structure to deploy the filter. As above, the amount of filter deployed may be varied depending on patient anatomy by varying how much of the frame to expose. In a variation of this embodiment, shown in FIG. 18A, the filter 113 instead could be occlusive to occlude the aorta during valve delivery rather than to filter the blood. In a variation of this embodiment, shown in FIG. 18B, the filter 113 is shaped to extend distally in the aorta. In this embodiment, the filter has a greater surface area and potentially has a lower effect on flow rate.

In any of the scenarios shown in FIGS. 15A-18B, an occlusion balloon 129 may be attached to the sheath 110 to block the accessed carotid artery. Additionally, during the step of filter retrieval after the valve is delivered, passive or active aspiration may be applied to the access sheath via Y-arm 112 to minimize the risk of embolic debris traveling to the downstream vessels. Optionally, irrigation may also be applied to assist in washing out loose debris, either through a channel in the sheath or via a separate irrigation catheter. As described above, the occlusion balloon 129 and aspiration and/or irrigation functions are not needed during the procedure as the aortic filter protects the access vessel; however, as described above, occlusion, aspiration and/or irrigation functions may be included in the system to provide protection during filter deployment and retrieval.

In all of the scenarios shown in FIGS. 15A-18B, the embolic filter material may be a perforated polymer film, a woven or knitted mesh material, or other material with a specific porosity. In an embodiment, the filter material porosity is between 80 and 150 microns. In an embodiment, the filter material porosity is between 100 and 120 microns. In an embodiment, the filter material is coated with heparin or other anti-coagulation agent, to prevent thrombus formation on the material during the procedure.

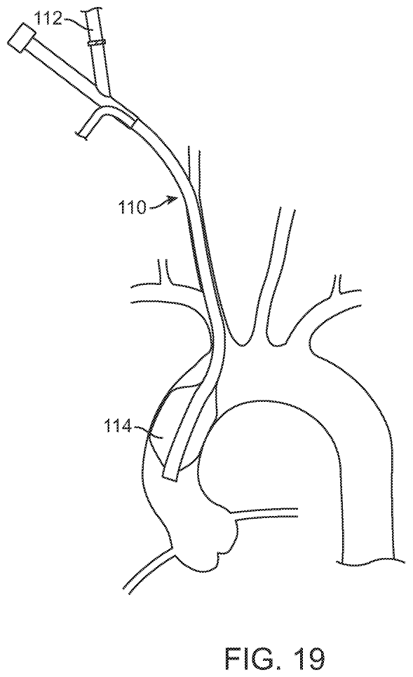

In another embodiment, as shown in FIG. 19, the access sheath 110 has an aortic occlusion element 114. The occlusion element is sized and shaped to occlude the ascending aorta. During use, the access sheath 110 is introduced via the right or left carotid artery and the distal portion is positioned in the ascending aorta. A pre-dilation balloon is positioned across the valve. Prior to pre-dilation of the valve, the heart flow is stopped or slowed significantly, e.g. via rapid pacing or atropine, and the occlusion element 114 is inflated or expanded to occlude the ascending aorta. The valve pre-dilation step is then performed without risk of distal emboli. Prior to deflation of the occlusion element 114, aspiration may be applied to the ascending aorta via the side arm 112 of the sheath 110. Optionally, irrigation may also be applied to assist in washing out loose debris, either through a channel in the sheath or via a separate irrigation catheter.

After the occlusion element 114 is deflated, the heart flow may be resumed. Next, the valve is positioned for implantation. As with the previous step, the heart flow is stopped or slowed significantly, e.g. via rapid pacing or atropine, and the occlusion element 114 is inflated or expanded to occlude the expanding aorta. The valve implantation step is then performed without risk of distal emboli. Prior to deflation of the occlusion element 114, aspiration may be applied to the ascending aorta via the side arm 112 of the sheath 110. The occlusion element is then deflated and heart flow is resumed with the newly implanted valve in place. The balloon material could be formed to create a non-compliant, complaint or semi-compliant structure. The balloon may be formed from PET, Silicone, elastomers, Nylon, Polyethylene or any other polymer of co-polymer.

In this configuration, the occlusion element 114 may be a balloon, which is expanded by inflation with a fluid contrast media. In this configuration, the sheath includes an additional inflation lumen which can be connected to an inflation device. Alternately, the occlusion element may be a mechanically expandable occlusion element such as a braid, cage, or other expandable mechanical structure with a covering that creates a seal in the vessel when expanded.

In another configuration, shown in FIGS. 20A and 20B, a first access sheath 110 is deployed transcarotidly (i.e., via the carotid artery, which may be inclusive of the internal carotid artery, external carotid artery, and/or the common carotid artery) into the artery so as to provide access to the aortic valve. The first access sheath 110 is configured as described above so that it can be used to provide cerebral embolic protection and to introduce a guide wire 119 into the vasculature and across the aortic valve. In addition, a second access sheath 1805 is introduced via alternate access site to access the aortic annulus from the other side, for example a transapical access site into the left ventricle. The second access sheath 1805 can be used to introduce a delivery system 400 which has been configured for transapical access for implanting the prosthetic valve 405. In this embodiment, the second access sheath 1805 may first be used to introduce a snare device 1810 that is configured to grasp or otherwise snare the distal end 1815 of the guide wire 119 that was inserted through the first access sheath 110, as in FIG. 20A. Alternately, as in FIG. 20B, the snare 1810 may be introduced via the first access sheath 110 and the guidewire 119 introduced via the second access sheath 1805. Irrespective of which end the guidewire was introduced or snared, the snare may be pulled back so that both ends of the guidewire may be secured externally. Such a double-ended securement of the guidewire 119 provides a more central, axially oriented and stable rail for placement of the prosthetic valve 405 than in a procedure where the guidewire distal end is not secured. The prosthetic valve 405 can then be positioned over the guidewire 119 via the second access sheath 1805, and deployed in the aortic annulus. In this embodiment, the first sheath 110 may be smaller than the second access sheath 1805, as the first sheath 110 does not require passage of a transcatheter valve.

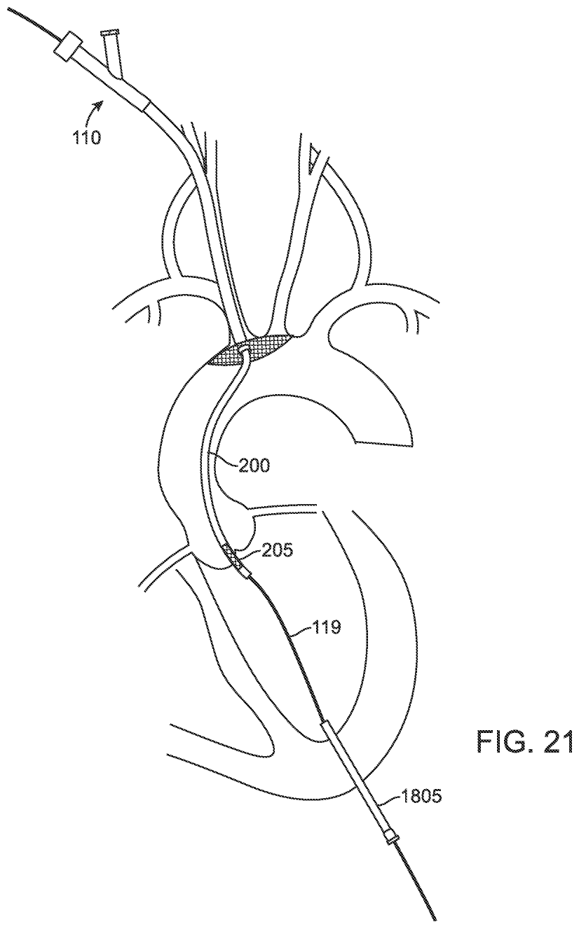

In a variation of the embodiment of FIGS. 20A and 20B, a transcarotid valve delivery system 200 and valve 205 may be delivered via the first sheath 110, as shown in FIG. 21. An advantage of this approach is that it requires a smaller puncture in the apex of the heart than the approach of FIGS. 20A and 20B. However a larger sheath is required in the first, access site. In this method embodiment, the first access site is shown as a transcarotid access in FIGS. 20 and 21, but may also be a sub-clavian or transfemoral access site.

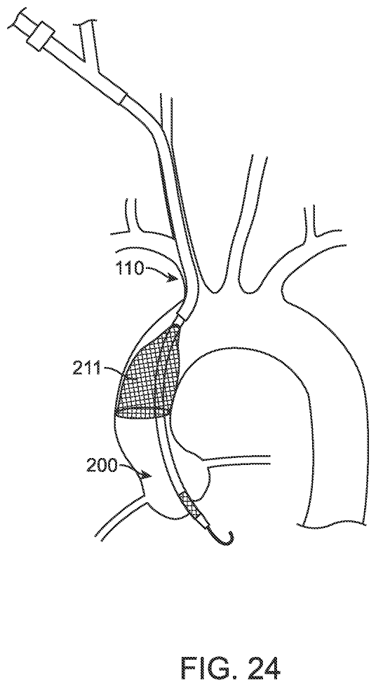

In another embodiment, the transcarotid valve delivery system also includes distal embolic protection elements. A shown in FIG. 24, the valve delivery system 200 includes an expandable filter element 211 that is sized and shaped to be expanded across the ascending when the valve delivery system 200 is positioned to deploy the valve in the desired location. Deployment of the filter may be accomplished by a retractable sleeve on the outside of the sheath, which, when retracted, exposes the expandable filter. Alternately, the filter may be pushed forward by means of a wire frame or structure to deploy the filter. The filter element may be affixed to the pusher sleeve 230 of the valve delivery system, or it may be affixed to a movable outer sleeve so that it can be independently positioned with respect to the valve. In the latter variation, the filter may be positioned and expanded before the valve has crossed the native valve location, thus protecting downstream flow from distal emboli during the crossing step of the procedure.

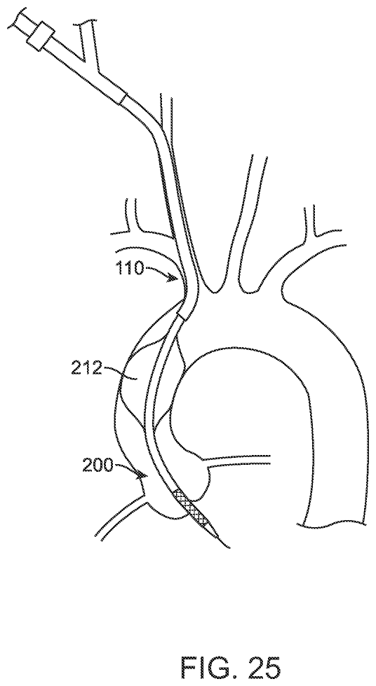

In a variation of this embodiment, as shown in FIG. 25, the valve delivery system 200 includes an occlusion element 212 which is sized and shaped to occlude the ascending when the valve delivery system 200 is positioned to deploy the valve in the desired location. The occlusion element 212 may be a balloon, which is expanded by inflation with a fluid contrast media. In this configuration, the valve delivery system 200 includes an additional inflation lumen which can be connected to an inflation device. Alternately, the occlusion element may be a mechanically expandable occlusion element such as a braid, cage, or other expandable mechanical structure with a covering that creates a seal in the vessel when expanded. The occlusion element may be affixed to the pusher sleeve 230 of the valve delivery system, or it may be affixed to a movable outer sleeve so that it can be independently positioned with respect to the valve.

If the access to the carotid artery was via a surgical cut down, the access site may be closed using standard vascular surgical techniques. Purse string sutures may be applied prior to sheath insertion, and then used to tie off the access site after sheath removal. If the access site was a percutaneous access, a wide variety of vessel closure elements may be utilized. In an embodiment, the vessel closure element is a mechanical element which include an anchor portion and a closing portion such as a self-closing portion. The anchor portion may comprise hooks, pins, staples, clips, tine, suture, or the like, which are engaged in the exterior surface of the common carotid artery about the penetration to immobilize the self-closing element when the penetration is fully open. The self-closing element may also include a spring-like or other self-closing portion which, upon removal of the sheath, will close the anchor portion in order to draw the tissue in the arterial wall together to provide closure. Usually, the closure will be sufficient so that no further measures need be taken to close or seal the penetration. Optionally, however, it may be desirable to provide for supplemental sealing of the self-closing element after the sheath is withdrawn. For example, the self-closing element and/or the tissue tract in the region of the element can be treated with hemostatic materials, such as bioabsorbable polymers, collagen plugs, glues, sealants, clotting factors, or other clot-promoting agents. Alternatively, the tissue or self-closing element could be sealed using other sealing protocols, such as electrocautery, suturing, clipping, stapling, or the like. In another method, the self-closing element will be a self-sealing membrane or gasket material which is attached to the outer wall of the vessel with clips, glue, bands, or other means. The self-sealing membrane may have an inner opening such as a slit or cross cut, which would be normally closed against blood pressure. Any of these self-closing elements could be designed to be placed in an open surgical procedure, or deployed percutaneously.

In an alternate embodiment, the vessel closure element is a suture-based vessel closure device. The suture-based vessel closure device can place one or more sutures across a vessel access site such that, when the suture ends are tied off after sheath removal, the stitch or stitches provide hemostasis to the access site. The sutures can be applied either prior to insertion of a procedural sheath through the arteriotomy or after removal of the sheath from the arteriotomy. The device can maintain temporary hemostasis of the arteriotomy after placement of sutures but before and during placement of a procedural sheath and can also maintain temporary hemostasis after withdrawal of the procedural sheath but before tying off the suture. U.S. patent application Ser. No. 12/834,869 entitled “SYSTEMS AND METHODS FOR TREATING A CAROTID ARTERY”, which is incorporated herein by reference in its entirety, describes exemplary closure devices and also describes various other devices, systems, and methods that are related to and that may be combined with the devices, systems, and methods disclosed herein.

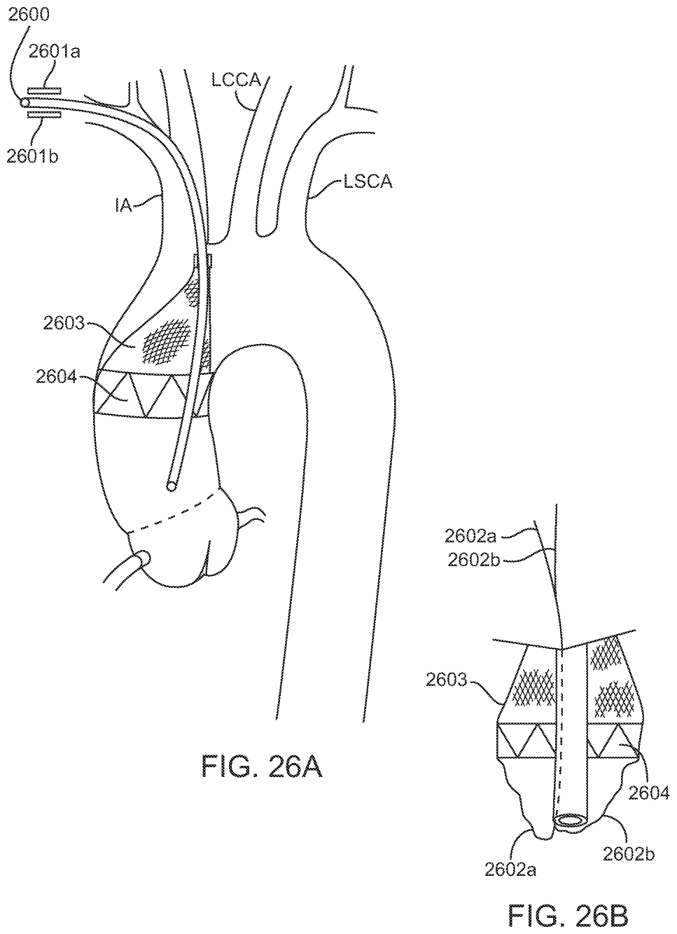

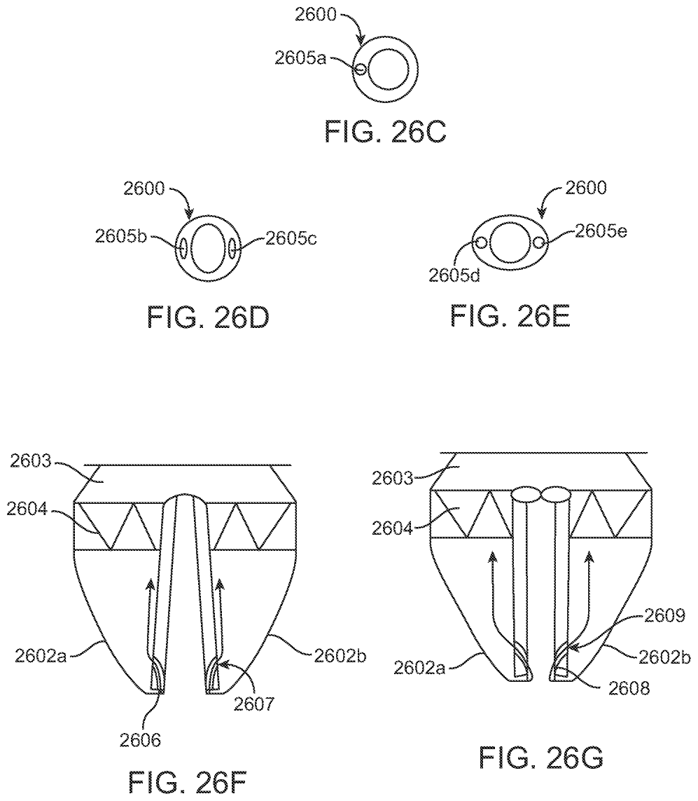

In another embodiment, shown in FIGS. 26A-26E, an aortic filter 2603 is configured as a cone shape or similar to a cone shape with an expandable distal band 2604, which is attached to the aortic filter 2603 such that the distal band 2604 extends at least partially around or is positioned within the aortic filter 2603. As shown in FIGS. 26A-26E, the aortic filter 2603 has a proximal end connected to an Inner Delivery Catheter (IDC) 2600, which has a proximal end positioned outside the patient's body. The IDC 2600 is an elongated body that can have one or more internal lumens, such as dual internal lumens. The IDC 2600 may be surrounded by an outer sheath 2601a,b (shown as a cross-section in FIG. 26A). The outer sheath 2601 can correspond to the arterial sheath 110 described herein. One or more pull wires 2602a and 2602b (shown in the partial view of FIG. 26B) connect to the aortic filter 2603. Distal portion of the pull wires 2602a and 2602b are connected to a distal portion of the aortic filter 2603. The pull wires 2602a and 2602b are also positioned inside a distal region of the IDC 2600 such as within a dual lumen.

As shown in the FIG. 26C, the outer lumen of the IDC 2600 can have a single round aperture 2605a in which both pull wires 2602a and 2602b may be positioned. Alternatively, as shown in FIG. 26D, the outer lumen of the IDC 2600 can have two elongated apertures 2605b and 2605c, or as shown in FIG. 26E, two round apertures 2605d and 2605e, such that each of the pull wires 2602a and 2602b is positioned in a separate aperture.

As shown in FIGS. 26F and 26G, different configurations of the pull wires 2602a,b within the lumen of the IDC 2600 may be used. The IDC 2600 may include one or two distal openings proximal to the distalmost opening 2606. As shown in FIG. 26F, the IDC 2600 may include a distal opening 2607 in communication with the outer lumen, such that the pull wires 2602a,b may be connected to the distal end of the aortic filter 2603 and extend back into the distalmost opening 2606 and inner lumen of the IDC 2600, and through the distal opening 2607 to pass through the outer lumen and exit the IDC 2600. Alternatively, as shown in FIG. 26G, the IDC 2600 may include a first distal opening 2608 and a second distal opening 2609 in the outer lumen which form a transverse passageway, such that the pull wires 2602a,b may be connected to the distal end of the aortic filter 2603 and extend back into a first distal opening 2608 of the IDC 2600, through the transverse passageway, and out a second distal opening 2609 to exit the IDC 2600.

The aortic filter 2603 can be collapsed using an outer sheath 2601a,b and/or pull wires 2602a,b. The pull wires 2602a,b can be connected to the distal end of the aortic filter 2603 and extend back down inside the distal end of the IDC 2600. A dual lumen IDC 2600 with separate lumen 2605a-e for the pull wires 2602a,b may be used. The use of pull wires 2602a,b, or the connection of the distal end of the aortic filter 2603 to the IDC 2600 allows the aortic filter 2603 to be collapsed around the IDC 2600 for removal. The collapsing occurs via a wrapping motion caused by the friction of the outer sheath 2601a,b as it is moved over the outer surface of the aortic filter 2603 and rotated inside the IDC 2600. The outer sheath 2601a,b may be placed over the aortic filter 2603 while the IDC 2600 is held in place.

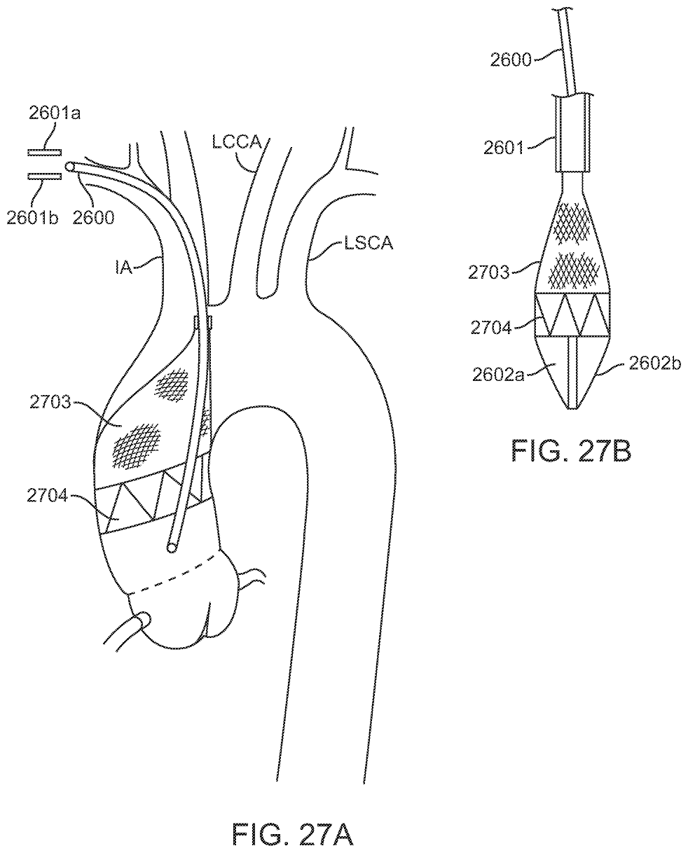

Referring now to FIGS. 27A-27C, in a variation of this embodiment, the aortic filter 2703 is configured as a cone with an expanding or expandable distal band 2704, which is attached to the aortic filter 2703 such that the distal band 2704 extends around or is positioned within the aortic filter 2703. The aortic filter 2703 has a proximal end connected to an Inner Delivery Catheter (IDC) 2600. The IDC 2600 may be surrounded by an outer sheath 2601 (show in cross section with reference numerals 2601a, 2601b). The aortic filter 2703 is configured to extend close to the aortic valve, and therefore have a larger filter area, as shown in FIG. 27A. To collapse the aortic filter 2703, pull wires 2602a,b (FIG. 27B) connected to the distal end of the aortic filter 2703 may be used (as shown in FIG. 27B) or the distal end of the aortic filter 2703 may be connected to the IDC 2600. Pull wires 2602a,b or connection of the aortic filter 2703 to the IDC 2600 may be used to collapse the aortic filter 2703 around the IDC 2600 for removal, using the friction of the outer sheath 2601 as it is moved over the outer surface of the aortic filter 2703 and rotated around the IDC 2600 to create a wrapping motion and facilitate collapse and removal. Either the pull wire closure configuration or the direct connection closure configuration 270 lb may be used to collapse and remove the aortic filter.

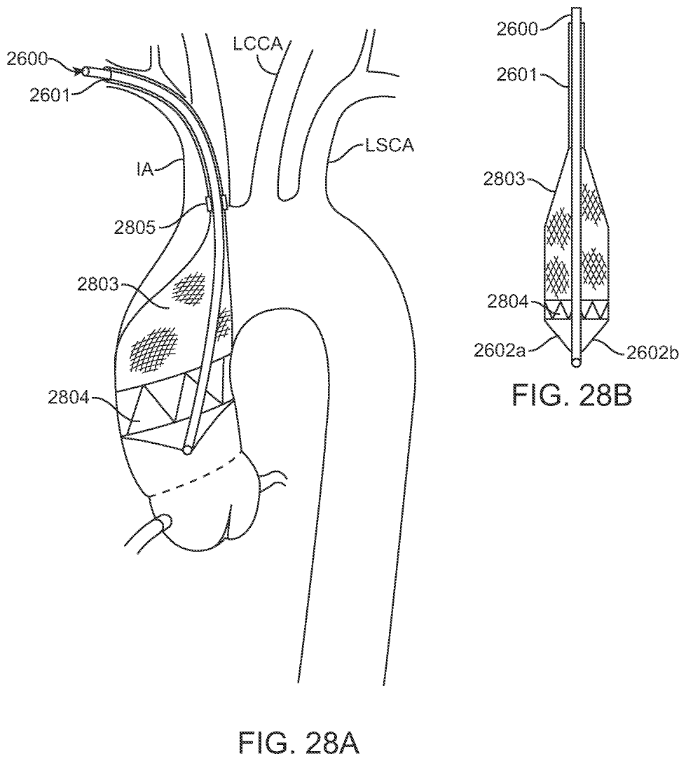

In another embodiment, shown in FIGS. 28A-28C, the aortic filter 2803 is configured as a cone with an expanding or expandable distal band 2804, which is attached to the aortic filter 2803 such that the distal band 2804 extends around or is positioned within the aortic filter 2803. The aortic filter 2803 has a proximal end connected to an Inner Delivery Catheter (IDC) 2600 via a lap joint 2805. The IDC 2600 may be surrounded by an outer sheath 2601. As shown in FIGS. 28A and 28B, the proximal end of the aortic filter 2803 is connected to the outer sheath 2601, and the distal end of the aortic filter 2803 is connected to the distal end of the IDC 2600. This configuration allows the aortic filter 2803 to be pulled closed or otherwise collapsed to a smaller size by moving the outer sheath 2601 proximally while holding the IDC 2600 stationary. This allows the aortic filter 2803 to be wrapped by direct rotation between the outer sheath 2601 and the IDC 2600, and does not require the sheath 2601 to be placed over the aortic filter 2803.

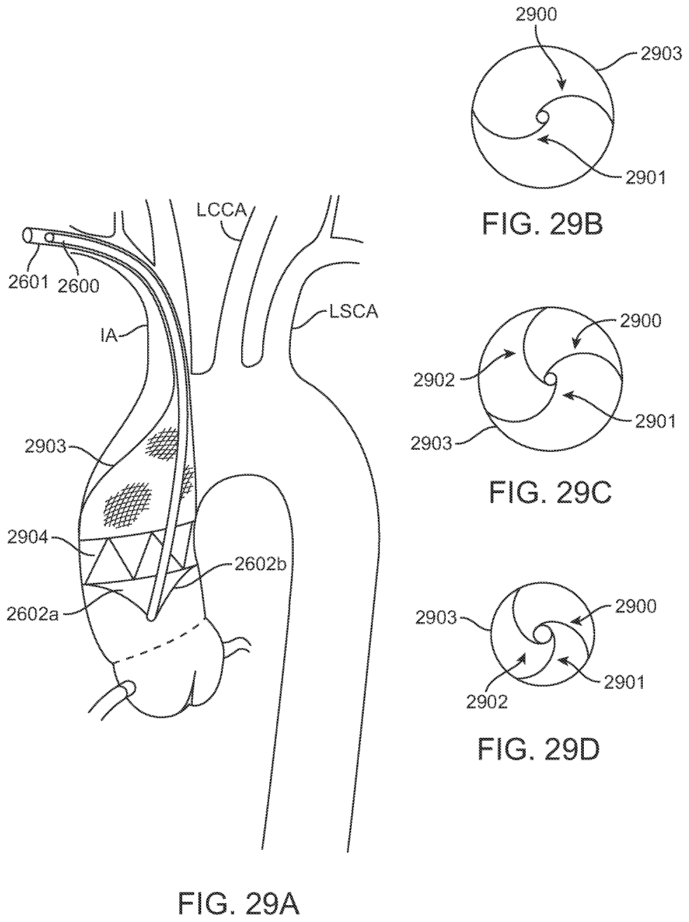

In a variation of this embodiment, shown in FIG. 29A, the aortic filter 2903 is configured as a cone with an expanding or expandable distal band 2904, which is attached to the aortic filter 2903 such that the distal band 2904 extends around or is positioned within the aortic filter 2903. The aortic filter 2903 has a proximal end connected to an Inner Delivery Catheter (IDC) 2600. The IDC 2600 may be surrounded by an outer sheath 2601. As shown in FIGS. 29B-29D, which show the aortic filter 2903 in cross-section, the distal end or region of the IDC 2600 may include radial struts 2900, 2901, and 2902, connected to the distal region of the aortic filter 2903 and to the central IDC 2600. The struts 2900, 2901, and 2902 can be tensed or wrapped to close or collapse the aortic filter 2903. Additionally or alternatively, the struts 2900, 2901, and 2902 can be compressed to provide a stabilizing force to the aortic filter 2903. The struts 2900, 2901, and 2902 can be at least partially spiral in shape or contour, which can help to center the IDC 2600 in the middle of the aortic vessel, as well as provide an outward force on the aortic filter 2903 that is not a single force point on the vessel wall, and provide the ability to break up and/or capture blood clots before the clots block off a large section of the surface of the aortic filter 2903.

The embodiments described above and shown in FIGS. 26A through 29D are illustrated and discussed as being introduced via the RCCA, however it should be appreciated that these embodiments may be adapted and/or modified for introduction via the LCCA. Any of the embodiments described herein can be delivered via the RCCA or the LCCA.

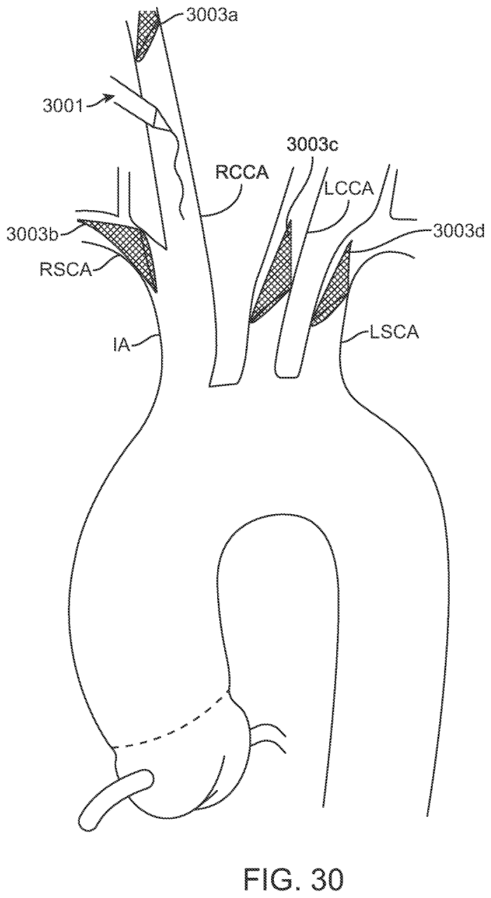

FIG. 30 shows a schematic view of the aortic arch and three arteries extending therefrom: the right common carotid artery (RCCA) including the right subclavian artery (RSCA) and the innominate artery (IA), the left common carotid artery (LCCA), and the left subclavian artery (LSCA). Multiple aortic filters 3003a, 3003b, 3003c, and 3003d can be deployed in one or more of the arteries to provide filter protection to a respective artery. For example, a filter 3003a is positioned in the RCCA. A transcatheter aortic valve replacement (TAVR) tool 3001 can be deployed into the RCCA distal to the filter 3003a via any of a variety of pathways. A filter 3003b is positioned in the RSCA. A filter 3003c is positioned in the LCCA. A filter 3003d is positioned in the LSCA. The filters 3003a-3003d can be deployed in the order of 3003a first, followed by 3003d, 3003c, and 3003b. Alternatively, the filters 3003a-3003d can be deployed in the order of 3003d first, following by 3003c, 3003b, and 3003a. Other orders of deployment are possible.

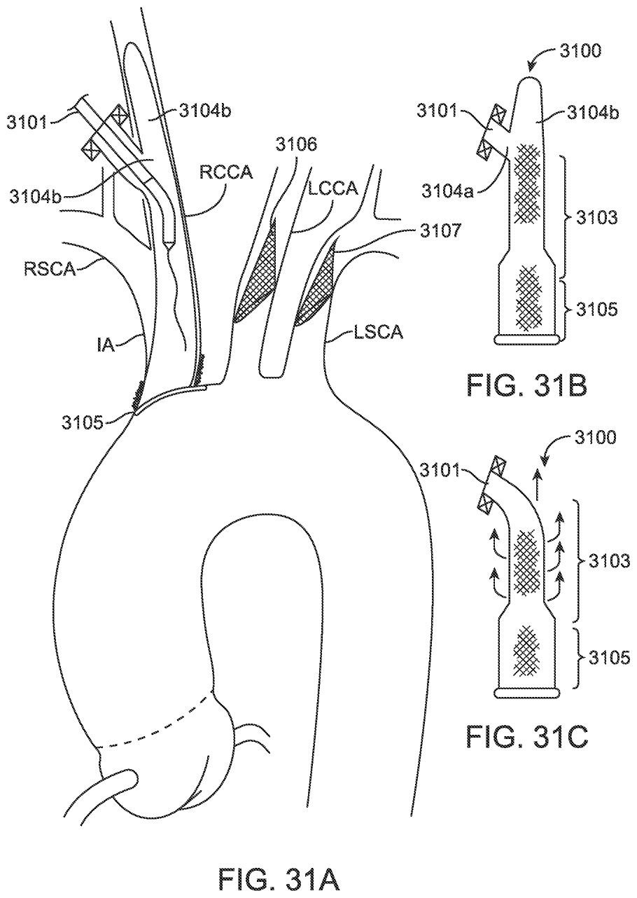

FIGS. 31A-31C, show an embodiment of an aortic filter system including a brachiocephalic filter 3100. The brachiocephalic filter 3100 has a main body 3103 with a distal neck region 3105, a first fork member 3104a, and a second fork member 3104b at a proximal region of the filter. The second fork member 3104b may have variable length or may be absent. The brachiocephalic filter 3100 may be deployed, for example, in the right RCCA such that the neck region 3105 contacts the RCCA and the main body 3103 does not contact the entire length of the RCCA. Additional filters 3106 and 3107 may be deployed in the LCCA and the LSCA respectively. Another embodiment of the brachiocephalic filter 3100 is shown in FIG. 31B. Yet another embodiment of the brachiocephalic filter 3100 is shown in FIG. 31C, without the second fork member 3104b. As illustrated in FIG. 31C, the brachiocephalic filter 3100 allows blood flow through the walls of the main body 3103 in the direction of the arrows shown.

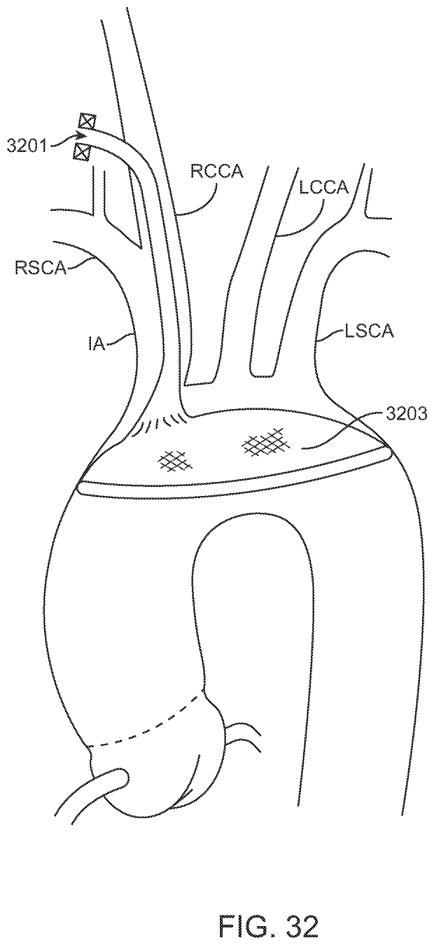

FIG. 32 shows another embodiment of a brachiocephalic filter 3201 having an elongate body and an aortic filter 3203. The elongate body extends through the RCCA (and may possibly occlude the RSCA) and is connected to the aortic filter 3203. The aortic filter 3203 is sized and shaped to sit in the aortic arch and protect the ostia of the arteries extending therefrom.

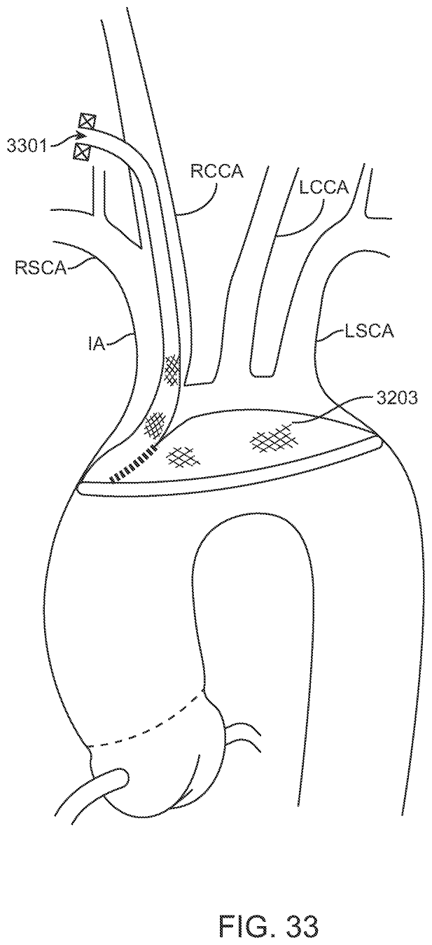

FIG. 33 shows yet another embodiment of a brachiocephalic filter 3301 having an elongate body, and a separate aortic filter 3303. The elongate body of the brachiocephalic filter 3301 extends through the RCCA and into the aortic arch, where it is connected to the edge of the aortic filter 3303. The aortic filter is sized and shaped to sit in the aortic arch and protect the ostia of the arteries extending therefrom.

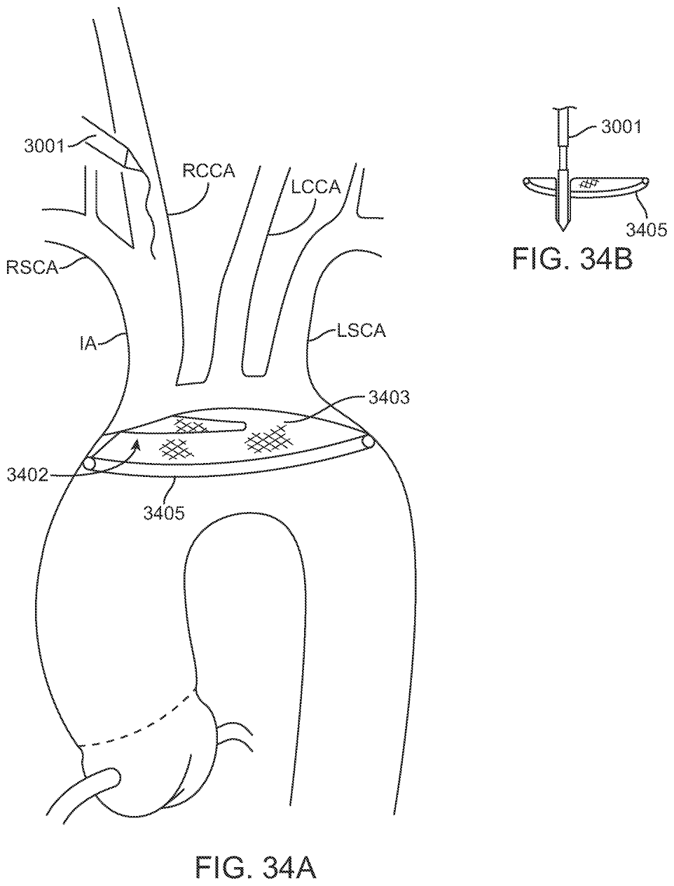

FIGS. 34A-34C are schematic views of an embodiment of an aortic filter 3403. A tool 3001 can enter the RCCA (via any of a variety of pathways), pass through the RCCA to the aortic arch, and pass through an aortic filter 3403 positioned in the aortic arch. The aortic filter 3403 is sized and shaped to sit in the aortic arch and protect the arteries extending therefrom. The aortic filter 3403 may include a distal sleeve 3405 disposed around an outer diameter of the aortic filter 3405, and a ring 3402 at the base of the sleeve 3405. The ring 3402 may be radiopaque. The sleeve 3405 may include for example Nitinol and/or urethane. The sleeve 3405 may serve as an external support. As shown in FIG. 34B, the sleeve 3405 can fit smoothly around the outer diameter of the TAVR tool 3001 after insertion.

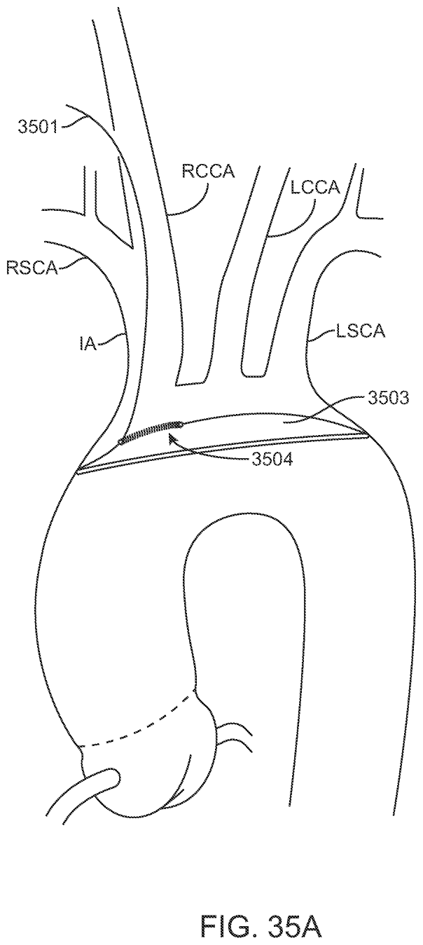

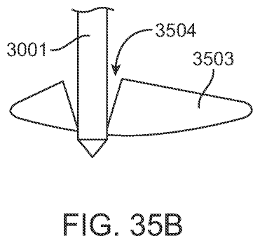

FIGS. 35A-35B are schematic views of an embodiment of an aortic filter 3503 attached to a support wire 3501. This embodiment is suitable for use in transcarotid and/or femoral access for example. The support wire 3501 extends through the RCCA and attaches to the aortic filter 3503 at one end of an access location 3504, as shown in FIG. 35A. The access location 3504 may include a tight mesh that remains closed until opened by insertion of a TAVR tool 3001. FIG. 35B illustrates insertion of the TAVR tool 3001 into the mesh of the access location 3504 and through the aortic filter 3503.

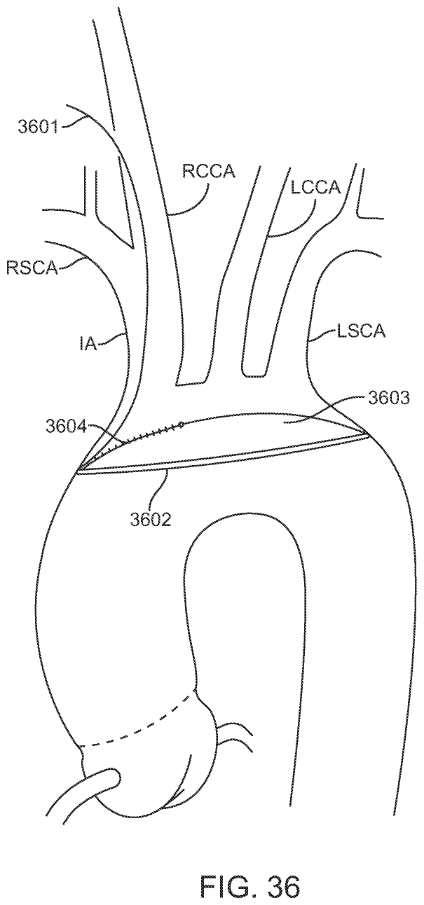

FIG. 36 is a schematic view of another embodiment of an aortic filter 3603 with a support wire 3601. This embodiment is also suitable for use in transcarotid and/or femoral access. The support wire extends through the RCCA and connects to the aortic filter 3603 and one edge of an access window 3604. The support wire 3601 may be made of any of a variety of materials. Non-limiting example materials include stainless steel, Nitinol, and aluminum. The aortic filter 3603 is sized and shaped to sit in the aortic arch and protect the arteries extending therefrom. The aortic filter 3603 includes a distal loop 3602 disposed around the diameter of the aortic filter 3603, and the access window 3604. The access window 3604 can have tighter porosity than the aortic filter 3603 and can loosen its porosity when a TAVR tool 3001 is inserted through the access window 3604. The access window 3604 can loosen its porosity to allow a TAVR tool 3001 to pass through the aortic filter 3603.

The embodiments described above and shown in FIGS. 32 through 36 are illustrated as being introduced via the RCCA, however it should be appreciated that these embodiments may be adapted and/or modified for introduction via the LCCA or other pathway.

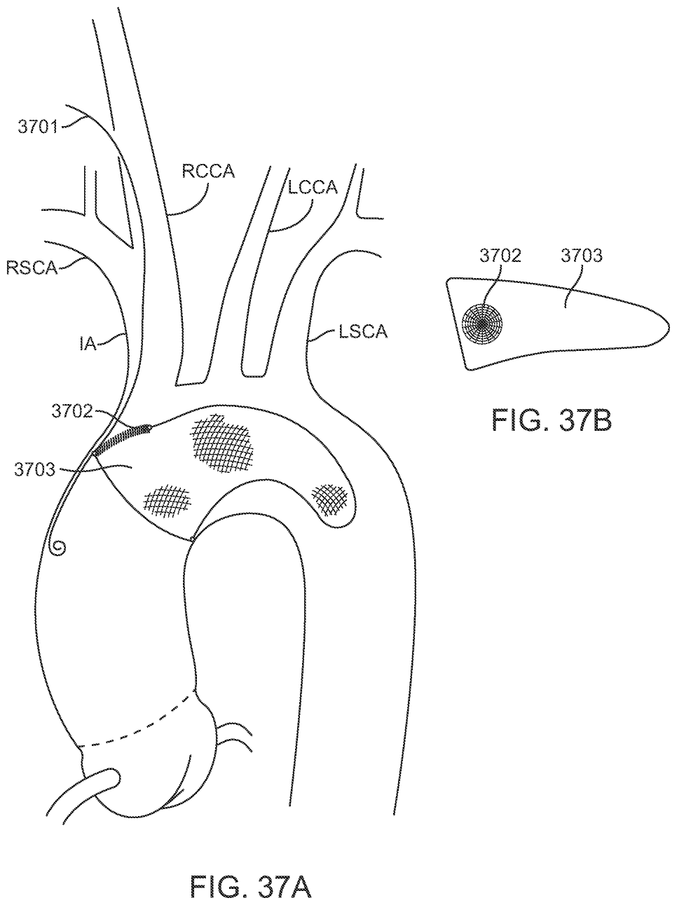

FIGS. 37A-37B are schematic view of yet another embodiment of an aortic filter 3703 with a support wire 3701. As shown in FIG. 37A, the support wire 3701 extends through the RCCA and connects to an access location 3702 on the aortic filter 3703. The aortic filter 3703 is sized and shaped to sit in the ascending aorta and expand through the aortic arch. FIG. 37B shows a cross-section of the aortic filter 3703 and access location 3702.

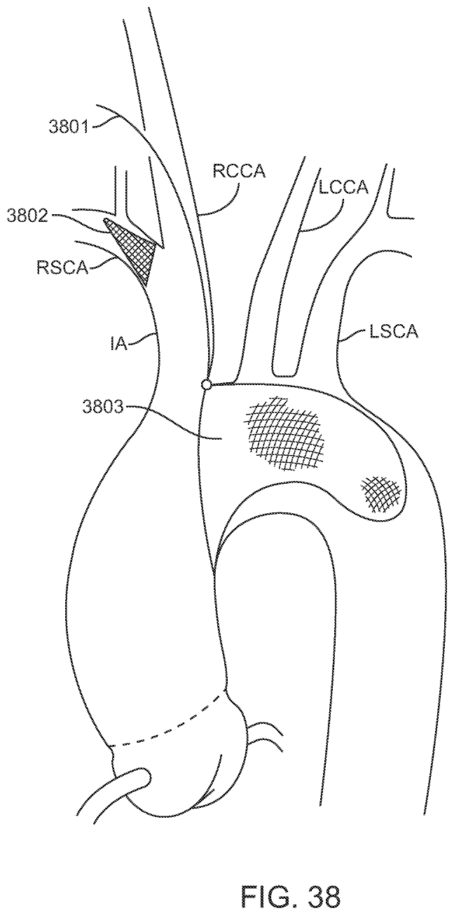

FIG. 38 is a schematic view of another embodiment of an aortic filter system. A support wire 3801 extends through the RCCA and connects to an aortic filter 3803. The aortic filter 3803 is sized and shaped to sit in the aortic arch and protect the LCCA and LSCA. Another filter 3802 is positioned in the RSCA. In this embodiment, neither the aortic filter 3803 nor the filter 3802 cover the RCCA.

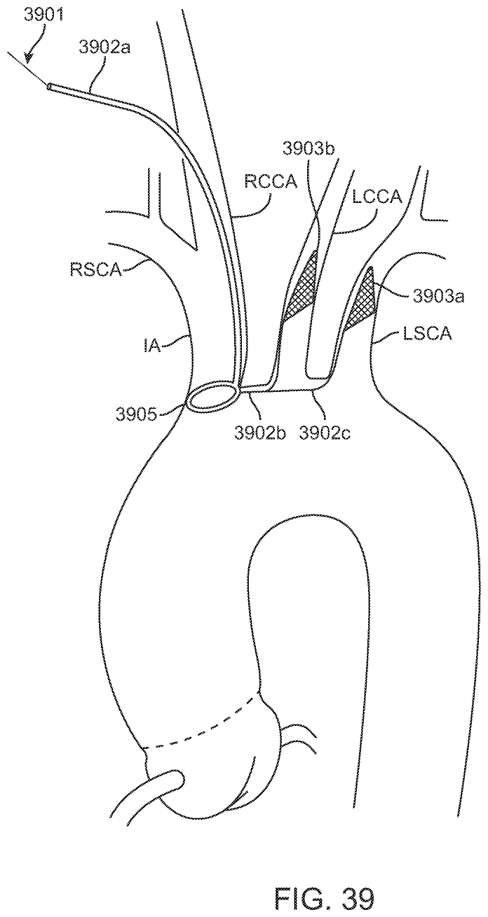

FIG. 39 is a schematic view of an aortic filter system. The filter system has a first elongate portion 3902a that is sized and shaped to extend through the RCCA. The first portion 3902a includes a lumen to receive a guidewire 3901. The first portion 3902a also includes an inflation port to allow inflation of the first portion 3902a and a loop structure 3905. The loop structure 3905 is inflatable and can create a seal around the delivery catheter. The seal is created between the delivery catheter and the inner wall of the innominate artery (IA). This seal can prevent embolic material from entering the RCCA around the delivery catheter. Inflation pressure can be modulated to allow axial translation of the delivery catheter while still maintaining a seal. The filter system has a second portion 3902b coupled to the first portion 3902a, such that the second portion 3902b is sized and shaped to extend through the LCCA, and a third portion 3902c which extends through the LSCA and is coupled to the second portion 3902b.

The first portion 3902a is coupled to the loop structure 3905, which sized and shaped to sit at the ostium of the RCCA. The first portion 3902a extends proximally through the RCCA and couples to the second portion 3902b. The second portion 3902b includes a filter 3903b attached to a bridge, and the filter 3903b is positioned inside the LCCA. The third portion 3902c also includes a filter 3903a that is positioned inside the LSCA and is connected to the second portion 3902b via a second bridge. The filter system is sized and shaped to be positioned in the RCCA, with a filter 3903b sized and shaped to be positioned in the LCCA and a filter 3903a sized and shaped to be positioned in the LSCA.

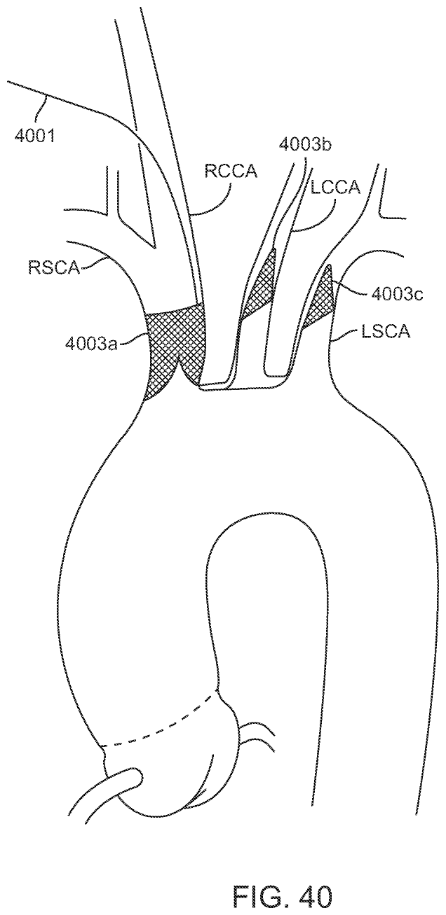

FIG. 40 is a schematic view of another embodiment of an aortic filter system. The system includes a guidewire 4001 having a first portion extending through the RCCA and connecting to a brachioaortic filter 4003a. The system further includes a filter 4003b connected to the guidewire 4001 by a first bridge and a filter 4003c connected to the guidewire 4001 by a second bridge. The brachioaortic filter 4003a is sized and shaped to sit at the ostium of the RCCA. The brachioaortic filter 4003a may be configured as a stent with an internal expandable filter. The filter 4003b is sized and shaped to be positioned in the LCCA. The filter 4003c is sized and shaped to be positioned in the LSCA.

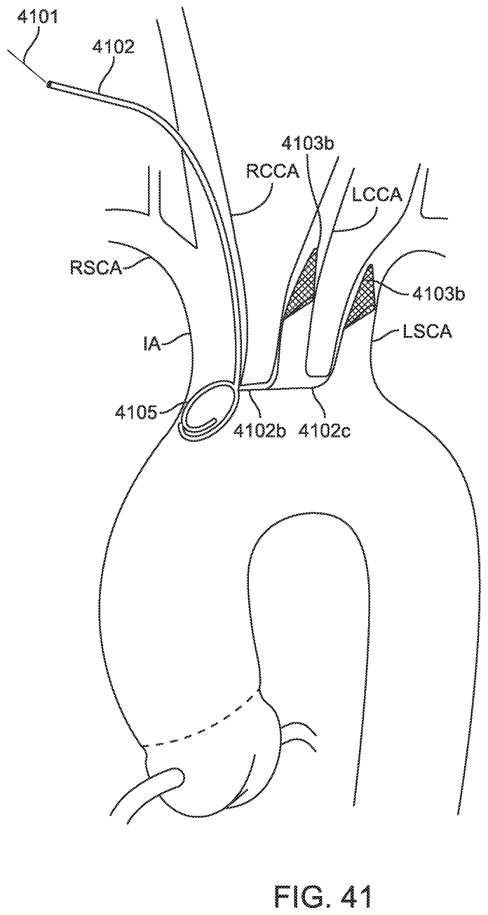

FIG. 41 is a schematic view of another embodiment of an aortic filter system. The filter system has a first elongate portion 4102a that is sized and shaped to extend through the RCCA. The first portion 4102a includes a lumen to receive a guidewire 4101. The first portion 4102a also includes an inflation port to allow inflation of the first portion 4102a and further includes hook structure 4105. The hook structure 4105 is inflatable and can create a seal around the delivery catheter (not shown). The seal is created between the delivery catheter and the inner wall of the innominate artery (IA.) This seal can prevent embolic material from entering the RCCA around the delivery catheter. Inflation pressure can be modulated to allow axial translation of the delivery catheter while still maintaining a seal. The filter system has a second portion 4102b coupled to the first portion 4102a, such that the second portion 4102b is sized and shaped to extend through the LCCA, and a third portion 4102c which extends through the LSCA and is coupled to the second portion 4102b.

The first portion 4102a is coupled to the hook structure 4105, which sized and shaped to sit at the ostium of the RCCA. The hook structure 4105 has two or more arms which wrap around each other to form a loop shape. The first portion 4102a extends proximally through the RCCA and couples to the second portion 4102b. The second portion 4102b includes a filter 4103a attached to a bridge, and the filter 4103a is positioned inside the LCCA. The third portion 3902c also includes a filter 4103b that is positioned inside the LSCA and is connected to the second portion 4102b via a second bridge. The filter system is sized and shaped to be positioned in the RCCA, with a filter 4103a sized and shaped to be positioned in the LCCA and a filter 4103b sized and shaped to be positioned in the LSCA.

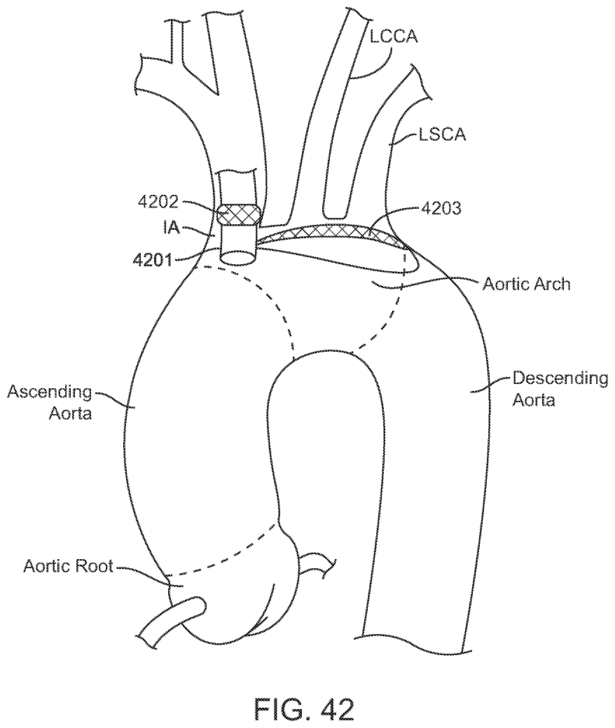

FIG. 42 shows schematic views of an embolic protection filter system. The system includes a transcarotid delivery sheath 4201 for a TAVR system. The delivery sheath 4201 optionally includes an occlusion balloon 4202 near a distal tip of the delivery sheath 4201. The balloon 4202 and delivery sheath 4201 are sized and shaped to be positioned within the innominate artery (IA). Inflation of the balloon 4202 prevents embolic material liberated from the aortic valve or the ascending aorta or aortic arch from entering the common carotid artery. A filter 4203 is attached to the distal end of the delivery sheath 4201. For access via the RCCA, the filter 4203 lays across the ostia of the LCCA and the LSCA. The filter 4203 prevents embolic material from entering the brain. Embolic materials are shunted past the aortic arch. During delivery, the filter 4203 may be collapsed in the forward position, distal to the body of the delivery sheath 4201.

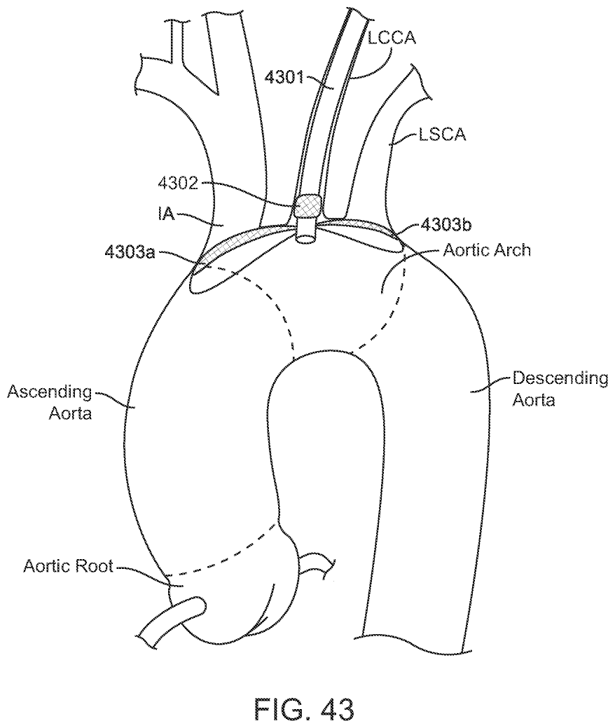

FIG. 43 shows schematic views of another embodiment of an embolic protection filter system. The system includes a transcarotid delivery sheath 4301 for a TAVR system. The delivery sheath 4301 optionally includes an occlusion balloon 4302 near the distal tip of the delivery sheath 4301. In any embodiment, the artery can be externally occluded such as by using a Rummel tourniquet. The balloon 4302 and delivery sheath 4301 are sized and shaped to be positioned within the LCCA. Inflation of the balloon 4302 prevents embolic material liberated from the aortic valve or the ascending aorta or aortic arch from entering the common carotid artery. Filters 4303a and 4303b are attached to the distal end of the delivery sheath 4301. For access via the LCCA, the filter 4303a lays across the ostium of the innominate artery (IA) and the filter 4303b lays across the ostium of the LSCA. The filters 4303a and 4303b prevent embolic material from entering the brain. Embolic materials are shunted past the aortic arch. During delivery, the filters 4303a and 4303b may be collapsed in the forward position, distal to the body of the delivery sheath 4301.

The embolic protection filter systems shown in FIGS. 42 and 43 may be secured in place via a movable thin-walled tube (not shown). The overall delivery profile of the system can thus be reduced compared to collapsing the full length of the filter against the outer surface of the delivery sheath. Embolic protection using these filter systems makes it possible to prevent embolic material from reaching the brain, since full filter baskets are not required. Embolic material is shunted past the ostia of the IA and LCCA and into distal vessel beds, where risk to the patient is substantially lower. The dual-filter configuration may be considered advantageous for certain applications, as the anatomy typically provides a relatively direct, straight path from the ostium of the LCCA to the aortic valve.

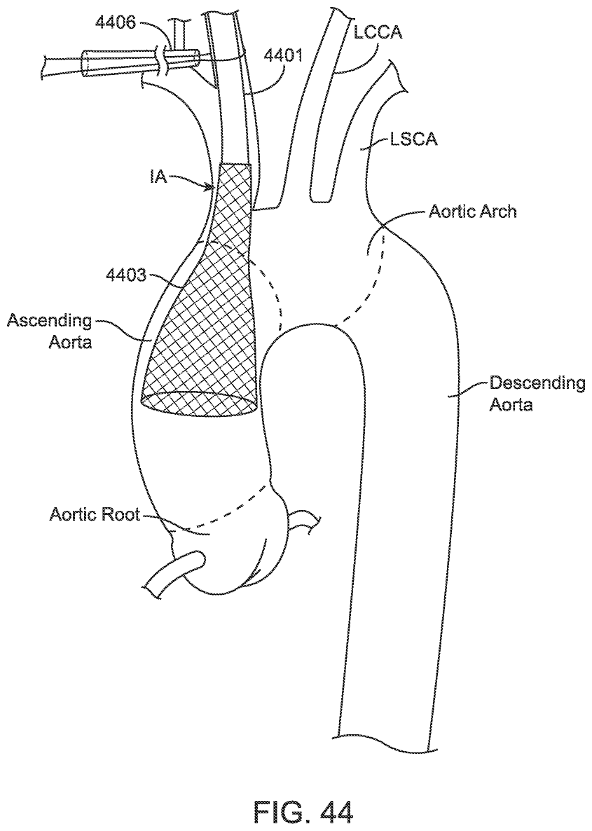

FIG. 44 shows a schematic view of an embodiment of an embolic protection filter system. As shown in FIG. 44, a valve delivery sheath 4401 is configured to terminate or be positioned within the innominate artery. An expandable filter 4403 extends into the descending aorta to filter any particulate matter which may be liberated during the aortic valve replacement procedure. A Rummel tourniquet 4406, or similar tourniquet or clamp device, may be used to secure the proximal portion of the valve delivery sheath 4401 within the common carotid artery. The Rummel tourniquet 4406 also reduces the likelihood that embolic material not captured by the expandable filter 4403 does not flow into the neurovasculature. The expandable filter 4403 may be constructed of a Nitinol mesh for example. The mesh may be heat set to self-expand and confirm to the inner surface of the aortic wall. The mesh may be reinforced with wires or struts to provide improved outward force, and to help secure the mesh against the wall of the aorta. The valve delivery sheath 4401 with expandable filter 4403 may be introduced into the aorta via either the right or left common carotid artery in a non-limiting example.

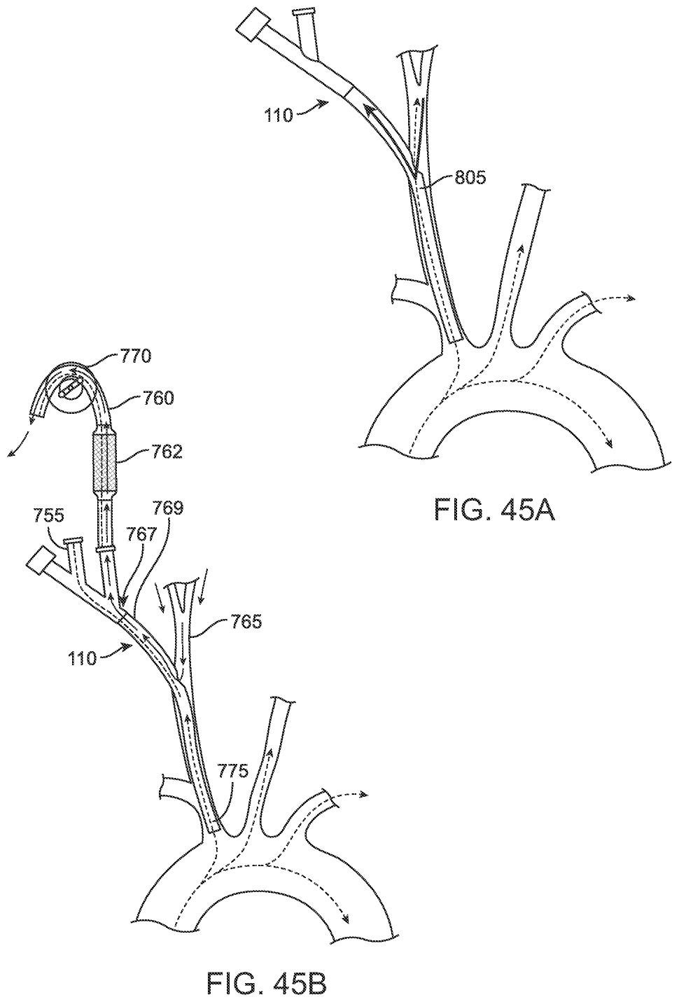

Referring now to FIGS. 45A-45B, in alternative embodiments, the arterial sheath 110 is configured to facilitate retrograde flow 805 from the ICA and ECA into the lower pressure venous circulation via an opening in the arterial sheath 110. Instead of shunting flow from the aorta to the distal CCA, the sheath draws blood from the distal carotid artery. Retrograde blood flow 805 from the ICA and ECA entrains blood from the contralateral side of the brain via the Circle of Willis and other collateral vessels, thus providing oxygenated blood to the ipsilateral side of the brain. Rather than providing a conduit to provide antegrade blood flow beyond the sheath into the distal CCA, this embodiment establishes retrograde blood flow 805. This may be a safe way to provide oxygenated blood to the ipsilateral circulation if there is carotid disease present at the bifurcation or in the ICA, which might embolize during antegrade blood flow in combination with vessel manipulation.

FIG. 45B shows an arterial sheath 110 deployed in the vasculature. A Y-arm 755 on the proximal region of the arterial sheath 110 is connected to a flow shunt 760 configured to reintroduce blood flow into the carotid artery at a location 765 upstream from the access point into the carotid artery. Retrograde blood flow from the ICA and ECA is represented by arrows. A second Y-arm 767 is fluidly connected to the shunt 760 and to a parallel lumen 769 that reintroduces blood from the shunt 760 into the artery at location 765. A filter element 762 may be included in the shunt 760 so that emboli generated during the procedure are not perfused into the cerebral artery.

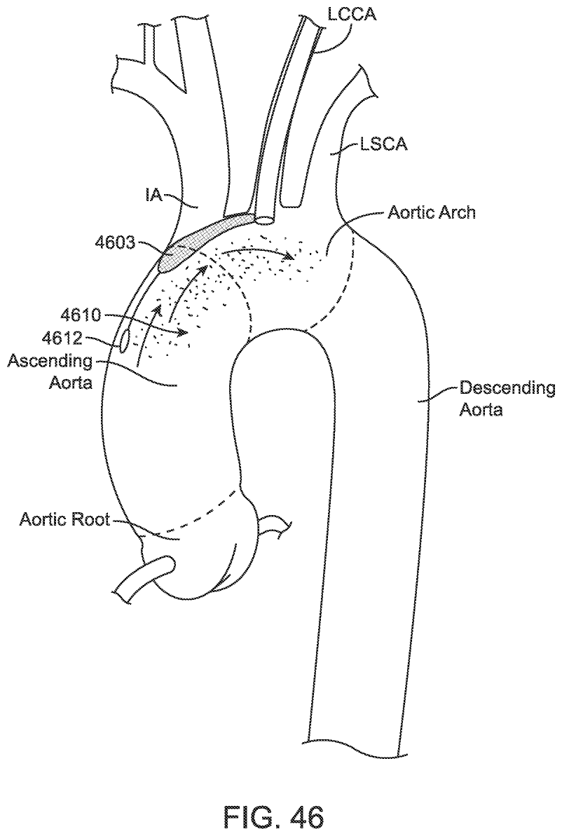

FIG. 46 shows another embodiment of an arterial sheath. As shown in the example of FIG. 46, the sheath is inserted into the left common carotid artery (LCCA.) At least one embolic protection element such as a filter 4603 extends toward the orifice of the innominate artery. The filter 4603 may be an integral part of the arterial sheath 110 (such as attached to a distal region of the sheath), or it may be a separate component such as coupled to an embolic protection system having an elongated element that can be delivered to the aorta via the arterial sheath 119. The filter 4603 does not necessarily directly occlude the innominate artery, or directly filter blood entering the innominate artery. Rather, the filter 4603 disrupts a blood flow pattern near the orifice of the innominate artery. The filter can be positioned at any location in the aorta such as at least partially in the ascending aorta.

The filter 4603 can have a size, shape, profile, or surface contour that is selected to modify the flow of blood through the aorta such as at the aortic arch. In this regard, the filter 4603 can have an outer surface that can be a solid surface or an interrupted surface (such as a mesh) that disrupts blood flow through the aorta. In an embodiment, the filter 4603 can include or be coupled to a separate component that is shaped to guide, direct, or disrupt blood flow in a particular manner such as in a specific direction or to generate a flow pattern. The filter 4603 can have a portion such as a fin or other shape that guides flow toward a location within the aorta. An embolic protection element such as a flow directing element 4612 can be positioned distal of the filter 4603 such as upstream of the filter. The flow directing element 4612 can direct blood flow toward the filter 4603, which can be positioned at any location within the aorta relative to the flow directing element 4612. The flow directing element 4612 can have a contoured surface, such as a concave surface, convex surface, angled surface (relative to a flow direction), curved surface, or combination thereof configured to direct blood flow in a desired direction. The filter 4603 can be positioned in the redirected flow pathway to capture debris, if present, in the redirected blood flow. The flow directing element 4612 or a second flow directing element can also or alternatively be positioned downstream of the filter 4603 such that the flow directing element further redirects blood flow away from an artery that branches off the aorta after the blood has flowed through and/or past the filter. In this manner, the flow directing element can redirect any debris that was not captured by the filter. The flow directing element 4612 can be attached to the filter or it can be a separate, detached component. In an embodiment, the flow directing element 4612 is also a filter. In another embodiment, the flow directing element is not a filter. The flow directing element 4612 can be delivered to a desired location through the sheath or it can be attached to the sheath such as to a distal region of the sheath. The flow directing element 4612 can also or alternatively be used in conjunction with any of the embolic protection devices described herein. For example, the flow directing element can be used in conjunction with a basket or a butterfly net, such as the filter 4403 shown in FIG. 44, wherein the flow directing element is positioned upstream or downstream of a capture element such as the filter 4403.

The filter can be positioned to extend into the lumen of the aorta or it may be positioned at least partially flush to a wall of the aorta. In an embodiment, as blood flows past and contacts the filter and/or the flow directing element, the filter or flow directing element interacts with blood flow to achieve a desired profile for blood flow. For example the components can cause blood to transition from a laminar flow to a turbulent flow, such as at or near the wall of the aorta. The disrupted blood flow can be such that any embolic material is caused to flow away from an orifice of an artery that branches off of the aorta. One or more capture filters (such as any of the filters describe herein) can be positioned in the aorta to capture such redirected blood flow such that a first filter or other element disrupts the blood flow in a manner that directs the disrupted blood for to one or more filters so as to capture any material in the blood flow.

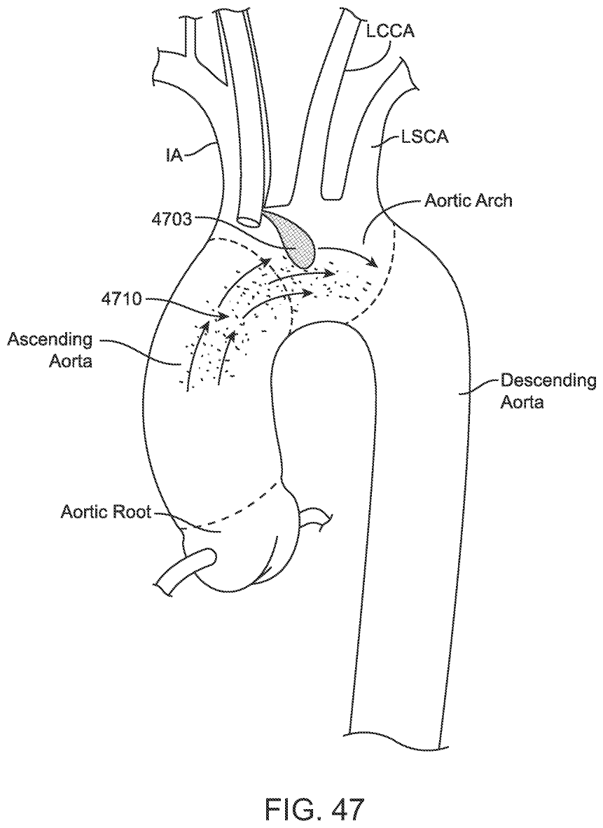

The presence of the filter 4603 may cause the flow to change, such as to transition from a laminar flow to a turbulent flow. The disrupted fluid flow pattern directs embolic material 4610 originating from the aortic valve away from the orifice of the innominate artery. The device may alternatively be configured to be introduced through the RCCA, as shown in FIG. 47. The filter 4603 can also be used in conjunction with a flow directing element such as the flow directing element 4612 described with respect to FIG. 47. The flow directing element can also be used with any type of embolic capture device that captures all or a portion of embolic debris in the aorta wherein a flow directing element is positioned upstream and/or downstream of such a embolic capture device.

Referring now to FIG. 47, a sheath may be inserted into the right CCA. A filter 4703 extends into the aortic arch. The filter 4703 directs embolic material 4710 disrupts the blood flow and directs embolic material away from the left CCA.

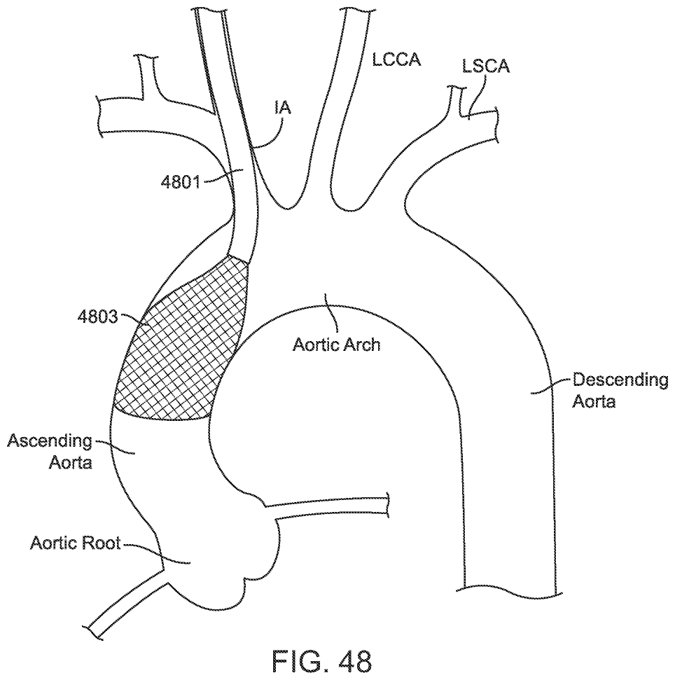

FIG. 48 shows another embodiment of an aortic filter 4803 and access sheath 4801. As shown in FIG. 48, the access sheath 4801 may be inserted through the IA via direct access, such as cut-down access, and advanced to the aorta such that a distal tip of the access sheath 4801 terminates or is otherwise positioned at, near, or in the ascending aorta. Alternatively, the access sheath 4801 may be inserted through the right CCA or left CCA via direct cut-down access and advance to the aorta. The distal tip of the access sheath 4801 may alternately terminate or otherwise be positioned at, near, or in the right CCA, left CCA, or IA.

With reference still to FIG. 48, the aortic filter 4803 may extend from the distal end of the access sheath 4801 and may be positioned within the ascending aorta. In some embodiments, if the access sheath 4801 is inserted via the left CCA, the aortic filter 4803 may also be deployed within a portion of the aortic arch. Placement of the aortic filter 4803 within the ascending aorta may prevent embolic material liberated from the aortic valve from being transported to the cerebral circulation and potentially causing stroke. The aortic filter 4803 may also prevent debris from being transported to the distal vascular beds.

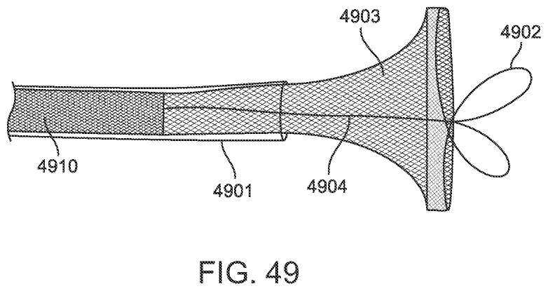

FIG. 49 shows an embodiment of a distal portion of an access sheath system closure filament sleeve. The system includes an outer capture sleeve 4901 formed of an elongated sleeve in which an inner shaft 4910 and a braided mesh filter 4903 (attached to the inner shaft 4910) are slidably and co-axially positioned. As described more fully below, a user can cause relative movement between the inner shaft 4910 (and attached mesh filter 4903) and the outer capture sleeve 4901 to release the braided mesh filter 4903 from being constrained by the outer capture sleeve 4901. This permits the braided mesh filter 4903 to transition between a smaller, constrained state to a larger, unconstrained state. Any of the filter described herein can vary in structure and can be, for example, braided, mesh, perforated, etc.

The inner shaft 4910 has an inner lumen having a diameter sized to allow slidable delivery of a sheath, such as a TAVR delivery sheath, therethrough. For example, the inner diameter of the inner shaft 4910 may be approximately 18F. The braided mesh filter 4903 may be constructed from a wire mesh, such as for example a shape-set Nitinol wire braid.

The braided mesh filter 4903 is attached to the inner shaft such as at a distal end or distal region of the inner shaft 4910. The braided mesh filter 4903 can be constructed from a shape set material, such as Nitinol wire braid. The braided mesh filter 4903 is configured such that it has a cone-shape or frusto-conical shape when unconstrained or unrestrained (as shown in FIG. 49.) The braided mesh filter 4903 is further configured to be collapsible into a smaller shape such as when the braided mesh filter 4903 is retracted and constrained into the outer capture sleeve 4901, as described more fully below.

With reference still to FIG. 49, a closure filament sleeve 4904 extends outwardly from the inner shaft 4910 and defines an internal lumen through which a closure filament 4902 (such as a length of wire) extends and protrudes. The closure filament 4902 is mechanically coupled to the braided mesh filter 4903 in a manner that permits a user to modify the shape of the braided mesh filter 4903 by manipulating the closure filament 4902. For example, the closure filament 4902 encircles a distal rim of the braided mesh filter 4903 and loops back into the lumen of the closure filament sleeve 4904. A user can withdraw or retract the closure filament 4902 into the closure filament sleeve 4904 to radially collapse the a distal portion or distal rim of the braided mesh filter 4903 closed and then retract the braided mesh filter 4903 into the capture sleeve 4910 (or slide the capture sleeve 4910 over the braided mesh filter 4903). In this manner, the braided mesh filter 4910 can be collapsed into a smaller size and constrained within the outer capture sleeve 4910.

In some non-limiting embodiments, the braided mesh filter 4903 may utilize a 32 mm diameter, one by two configuration braid comprised of approximately 144 individual wires, such as for example 0.002″ diameter Nitinol wires. The braid may be folded back on itself to provide a double-layer mesh. A double-layer mesh has advantages over a single-layer mesh, such as a smooth distal rim or edge with no exposed wire ends, a smaller effective pore size of the mesh, and/or increased structural rigidity. The wire diameter, wire count, and braid configuration of the mesh can be varied to adjust the stiffness/conformability and pore size of the braided mesh filter 4903. The braided mesh filter 4903 as shown in FIG. 49 is double-layer mesh, however the number of layers may be varied (for example, single-layer, quad-layer, etc.) depending on the desired characteristics of the braided mesh filter 4903. The structure of the braided mesh filter 4903 may be supplemented with additional support features, such as for example struts, hoops, loops, or other features configured to enhance the overall structural integrity and outward radial force of the braided mesh filter 4903, to increase stability within the aorta. Additionally and/or alternatively, the structure of the braided mesh filter 4903 may be supplemented with additional mesh materials having smaller pore sizes (for example, polyethylene terephthalate (PET) wires, fine wire nitinol mesh, etc.).

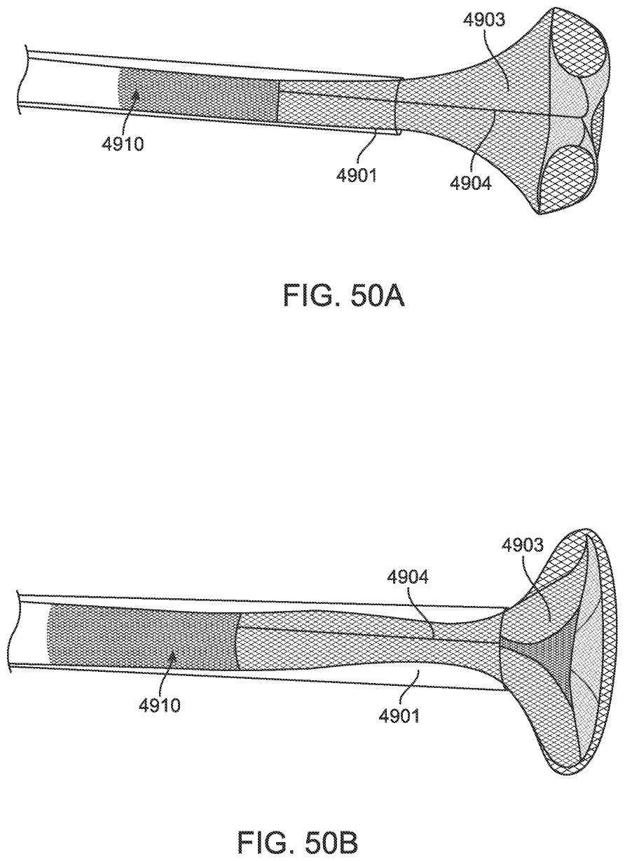

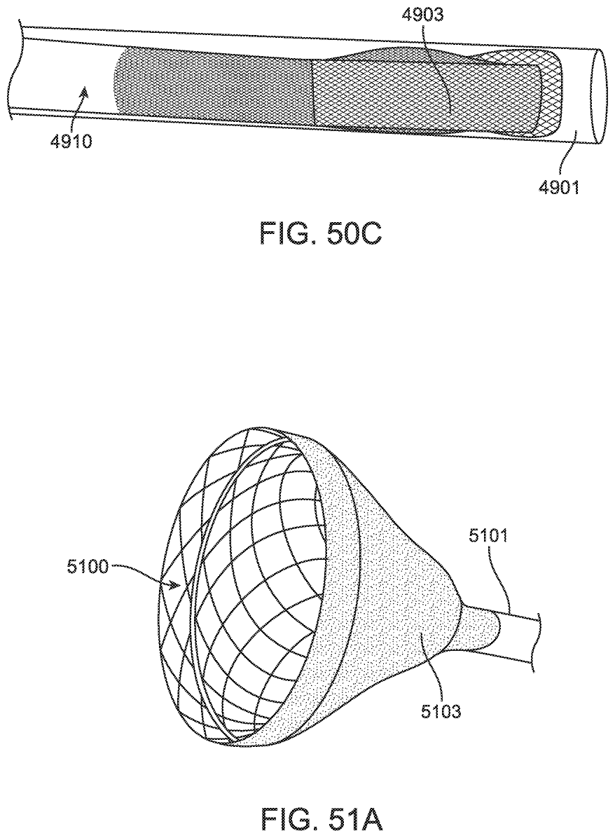

As shown in FIGS. 50A, 50B, and 50C, shortening, pulling, or otherwise actuating the closure filament via the closure filament sleeve 4904 can draw the distal opening of the braided mesh filter 4903 closed. The closure filament sleeve 4904 may be withdrawn to pull the closure filament 4902 and the attached distal portion of the braided mesh filter 4903 into the inner diameter of the inner shaft 4910, everting the braided mesh filter 4903 and reducing its outer profile. FIG. 50A shows the closure filament withdrawn via the closure filament sleeve 4904, thereby closing the distal end of the braided mesh filter 4903 and capturing any embolic material within the braided mesh filter 4903. Drawing the braided mesh filter 4903 closed can prevent captured embolic material from becoming liberated during collapse and withdrawal of the braided mesh filter 4903.

FIG. 50B shows the closure filament sleeve 4904 and the closure filament 4902 partially retracted to collapse and evert the braided mesh filter 4903. That is, the cone-shape of the braided mesh filter 4903 everts onto itself and collapses as the closure filament sleeve 4904 and the closure filament 4902 are retracted. FIG. 50C shows an outer capture sheath 4901 advanced over the collapsed filter cone 5003. After the braided mesh filter 4903 is collapsed or during collapse of the braided mesh filter 4903, the outer capture sleeve 4901 can be slid over the braided mesh filter 4903.

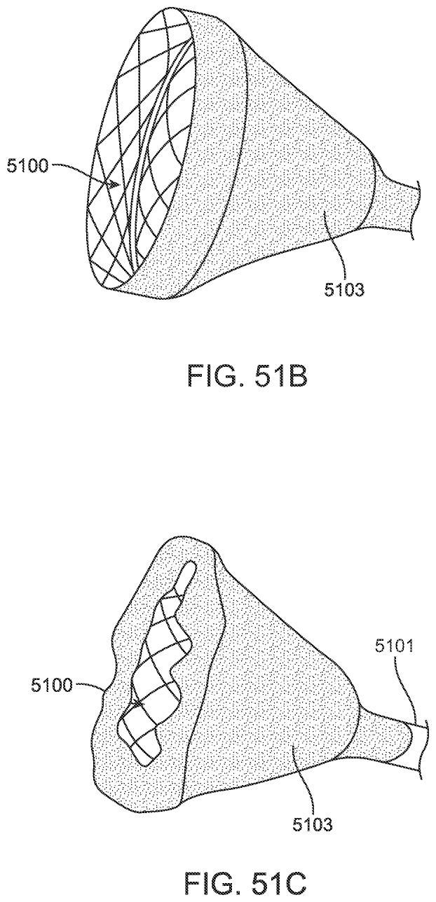

FIGS. 51A, 51B, and 51C show an embodiment of a filter assembly including a fabric mesh filter 5103 with braid support 5100. The fabric mesh filter 5103 may be porous, such as for example a fabric filter with pores of approximately 150 micrometers. The braid support 5100 may be a wire braid, for example, a Nitinol wire braid. The braid support 5100 is configured to provide a support lattice for a secondary filter membrane, such as the fabric mesh filter 5103. In some embodiments, a 0.010″ nitinol wire may be used to form the braid support 5100. As shown in FIG. 51B, the fabric mesh filter 5103 and braid support 5100 may be attached to the distal portion of an access sheath 5101. Advancing the braid support 5100 co-axially relative to the access sheath 5101 may increase the outward radial force that the structure applies to the vessel wall. Additionally and/or alternatively, the braid support 5100 may improve apposition of the fabric mesh filter 5103 against the aorta wall, and/or improve stability of the fabric mesh filter 5103 in the aorta.

As shown in FIG. 51C, retracting the braid support 5100 co-axially relative to the access sheath 5101 may reduce the outer dimensions of the assembly for ease of deployment or retrieval. For example, retraction of the braid support 5100 may evert the distal portion of the fabric mesh filter 5103, which may assist with retaining embolic material captured by the fabric mesh filter 5103. FIG. 51A shows the fabric mesh filter 5103 with the braid support 5100 attached to the inner surface of the fabric mesh filter 5103. FIG. 51B shows the braid support 5100 fully advanced, to provide added outward radial force to the fabric mesh filter 5103. FIG. 51C shows the braid support 5100 partially retracted, to partially evert the distal portion of the fabric mesh filter 5103 for particle capture and retrieval.

In some embodiments, the braid support 5100 may be configured to attach to the outer surface of the fabric mesh filter 5103, rather than to the inner surface of the fabric mesh filter 5103 as is shown in FIGS. 51A-51C. The braid support 5100 may alternatively be positioned between two layers of filter material, within a filter material, or any other suitable configuration. Additionally and/or alternatively, the fabric mesh filter 5103 may be configured to be pliable or to stretch, such as to enable sizing and/or expansion of the fabric mesh filter 5103. The pliability or stretch of the fabric mesh filter 5103 may also help to achieve better apposition against the wall of the aorta. Additionally and/or alternatively, Nitinol wire of a smaller diameter than that shown in the figures, for example 0.006″-0.008″ diameter, may be used to construct the braid support 5100. This may create a more pliant and/or flexible structure for better conformity of the braid support 5100 and fabric mesh filter 5103 to the aortic wall. It should be appreciated that other combinations of filter material and braid material, and/or combinations of flexible versus rigid filter or braid material, may be used to achieve the desired characteristics (e.g. pliability, conformity to aortic wall, etc.) of the filter and braid assembly. Additionally and/or alternatively, the fabric mesh filter 5103 may be configured to have an extended distal cylindrical shape, rather than a conical or frusto-conical funnel shape. Such an extended distal cylindrical shape may increase the surface contact of the fabric mesh filter 5103 with the wall of the aorta and improve the stability of the fabric mesh filter 5103 when positioned within the aorta.

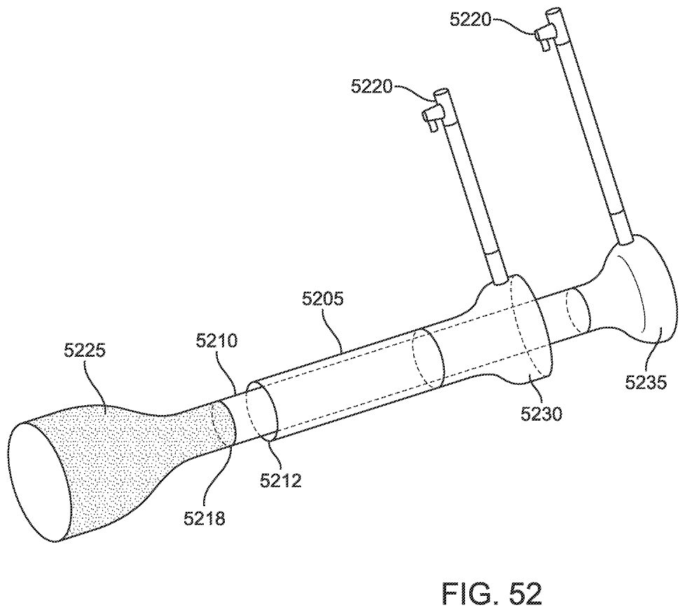

FIG. 52 shows an embolic protection system that includes a pair of elongated, coaxial, telescoping sheaths each having an internal lumen. The sheaths include an outer sheath 5205 and a co-axial inner sheath 5210 slidably positioned within the outer sheath 5205. The outer sheath 5205 has a proximal hub 5230 that can be grasped by a user to manipulate the outer sheath. Likewise, the inner sheath 5210 includes a proximal hub 5235 that extends proximally outward from the proximal end of the outer sheath 5205. Each hub can include a respective outlet line 5220 that can include a flow control device such as a stopcock.

With reference still to FIG. 52, a flexible filter structure 5225 is attached to a distal region of the inner sheath 5210 such as at a location 5218. There can be a clearance 5212 between the inner sheath 5210 and the outer sheath 5205. In an example embodiment, the clearance is at least 0.005 inch. The filter structure 5225 forms a filter basket that can be used to capture or divert embolic material within the aorta when the filter structure 5225 is deployed therein. The filter structure 5225 can be manufactured of any a variety of materials. In an embodiment the filter structure 5225 is constructed from a collapsible Nitinol braid that is mated to a secondary micro mesh material. The Nitinol braid provides structure and support to the filter structure. The braid can also facilitate expansion of the filter structure against the wall of the aorta when positioned therein. The micro mesh may be knitted or woven from a material such as PET or the like, for example. In another embodiment, the micro mesh is constructed from a solid sheet of material and perforated with holes. The hole or pore size of the micro mesh can vary and in an embodiment is in the range of 100 μm to 300 μm, or up to about 500 μm. The micro mesh is configured to permit passage of blood therethrough while still capturing embolic materials.

The inner catheter 5210 is sufficiently large to accommodate positioning a TAVR delivery catheter (such as an 18F or smaller catheter) therethrough. The outer catheter 5205 may be advanced distally relative to the inner catheter 5210 such that the outer catheter 5205 captures, collapses, or constrains the filter structure 5225 so as to facilitate delivery of the filter structure 5225 through a lumen of a blood vessel. Once the system has been delivered to a target location, such as at or near the aorta, the outer catheter can be proximally withdrawn relative to the inner catheter to permit the filter structure to expand to a desired shape.

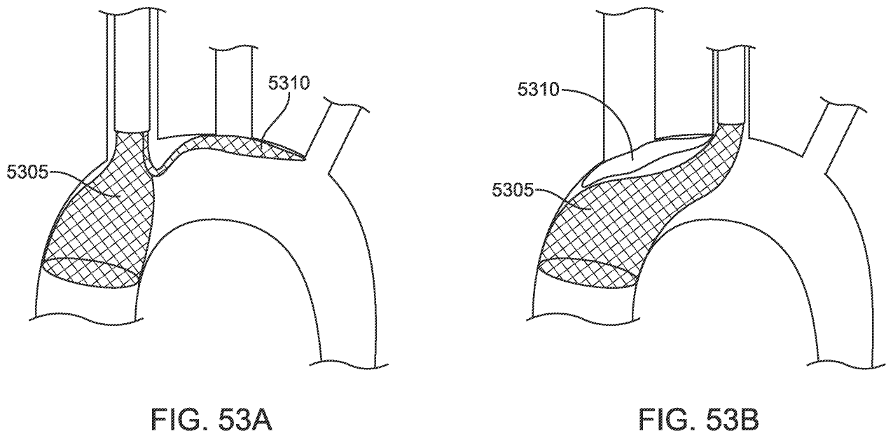

FIG. 53A shows a schematic view of a filter 5305 positioned within the aorta. The filter 5305 can have a cone configuration such as any of the cone-like configurations described herein. The system further includes a wing filter 5310 that can be positioned in the aorta relative to the filter 5305. When the filter 5305 is positioned in the ascending or transverse aorta, the wing filter 5310 can be both deployed and positioned to sit across or otherwise cover an orifice of one or more arteries at branch off the aortic arch. The filter 5305 serves as a primary filter that provides primary embolic protection by capturing embolic debris in the aorta. The wing filter 5310 serves as a secondary protective element that can capture any embolic debris not captured by the filter 5305 or divert embolic debris away from the orifices of branch vessels of the aorta that lead to the brain. The embodiment of FIG. 53A and similarly the embodiment of FIG. 53B can be deployed from either the left carotid artery or the right carotid artery. The position of the wing filter 5310 can vary relative to the position of the filter 5305.

The pore size of the filter 5305 or the wing filter 5310 can be configured to optimize embolic capture of blood flow by the filter. The pore size of the filter 5305 can be adjusted or otherwise configured to allow for robust blood flow while still capturing embolic debris. The pore size of the wing filter 5310 can be configured to divert or guide embolic material away from an artery orifice that branches off the aorta. A smaller pore size of the wing filter 5310 can inhibit smaller embolic debris not captured by the filter 5305 from entering the branch arteries. It should be appreciated that the wing filter 5310 does not necessarily have to sit flush against an orifice of an artery. The wing filter 5310 can be configured to create or encourage a specific blood flow pattern such as to direct blood flow and any embolic particles downstream and away from orifices of the branch arteries. The pore size can differ between the filter 5305 and the wing filter 5310 such as to achieve a desired flow pattern of blood flowing therethrough. In any of the embodiments described herein, one or more of the orifices can have an asymmetric shape. The pore size of a filter may have a narrow width (such as 50 μm) that inhibits particles traveling through the filter, while also having a relatively long length (such as 800 μm) that provides additional cross-sectional area to decrease resistance to blood flow therethrough.

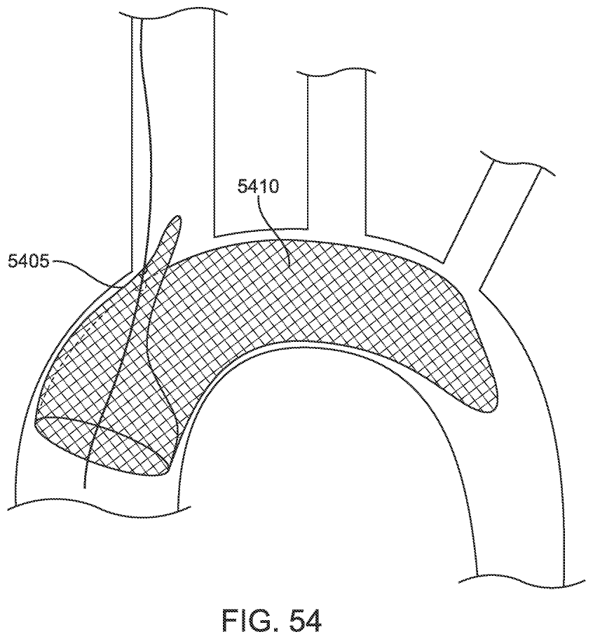

FIG. 54 shows another embodiment wherein a filter 5410 is sized and shaped to be positioned fully within the aortic arch such that the filter 5410 covers multiple orifices of the vessels branching off of the aortic arch, such as the innominate artery, the left carotid artery, and/or the left subclavian artery. The filter 5410 can be constructed to include multiple regions, wherein each region has a pore size and shape specific to that region and wherein the pore size and/or shape of one region may be different from another region. For example, one region may be a fine mesh having a pore size in the range of 50 μm to 100 μm. This region may be positioned at or across the superior aspect of the aortic arch to provide embolic protection to the branch arteries. The pores in this region may allow blood to cross the membrane and flow into the branch arteries. The pores may be sufficiently small to prevent emboli from passing through that region.

Another region of the filter 5410 may comprise a coarse mesh having a pore size in the range of 150 μm to 130 μm or larger. This region may cover the inferior portion of the aortic arch and the distal, enclosed section of the filter 5410. The relatively larger pore size permits blood to flow swiftly through the filter in that region. Embolic particles suspended in the blood may be carried with the blood distally, past orifices of the branch vessels. The particles would be captured by the filter to prevent them from flowing into distal vessel beds. Thus, the fine-mesh region of the filter 5410 positioned across the superior aspect of the aortic arch behaves as a flow diverter. It should be appreciated that the size, shape and quantity of each region of the filter 5410 can vary.

With reference still to FIG. 54, the system can include a frame 5405 coupled to the filter 5410. The frame 5405 may have an asymmetric shape and may be manufactured of the material such as a Nitinol braid. In an embodiment, the frame includes a rapid exchange port for a guidewire.

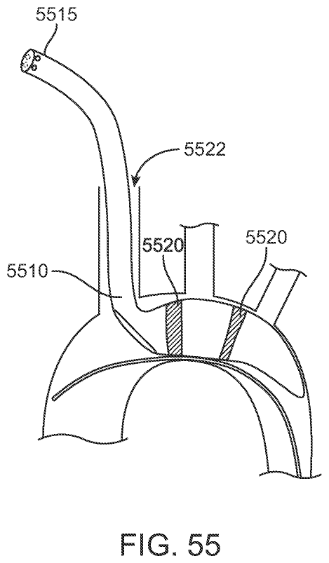

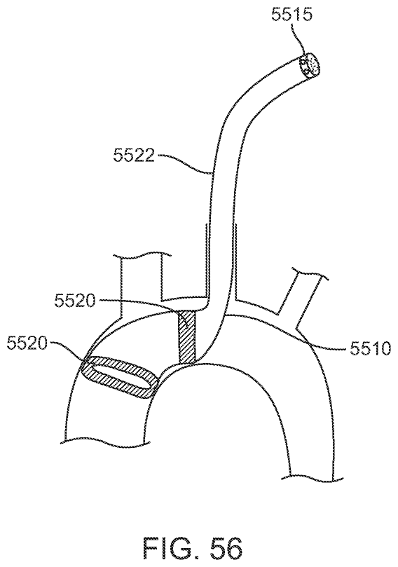

FIG. 55 shows another embodiment wherein a filter 5510 comprises a single piece of material, such as a single piece of braided tubing of Nitinol wire or the like. The filter 5510 has a distal region formed into a shape, such as a basket or cone shape, configured to capture embolic material while permitting blood to flow therethrough. The filter 5510 can be configured to optimize positioning for delivery from the right carotid artery or the left carotid artery as shown in FIGS. 55 and 56. The filter 5510 is configured to provide embolic protection for all arteries branching off the transverse aorta as well as providing protection to distal vessel beds. The filter 5510 may include one or more segments or regions 5520 that are configured to provide enhanced, outward, radial force so as to increase apposition against a wall of the aorta.

A proximal region 5522 of the filter 5510 can be positioned within the left or right carotid artery and expanded to a cross-section that conforms to the inner diameter of the carotid artery, such as a circular cross-section. The proximal section of the filter 5510 may exit through a side wall of the carotid artery. A section 5515 may be encapsulated by a thin layer of material, such as a polymer layer that prevents blood from exiting through open cells of the braid. The filter 5510 may be constructed with different core dimensions to facilitate filtering and flow diversion of particles so as to inhibit these particles from traveling into the cerebral circulation.

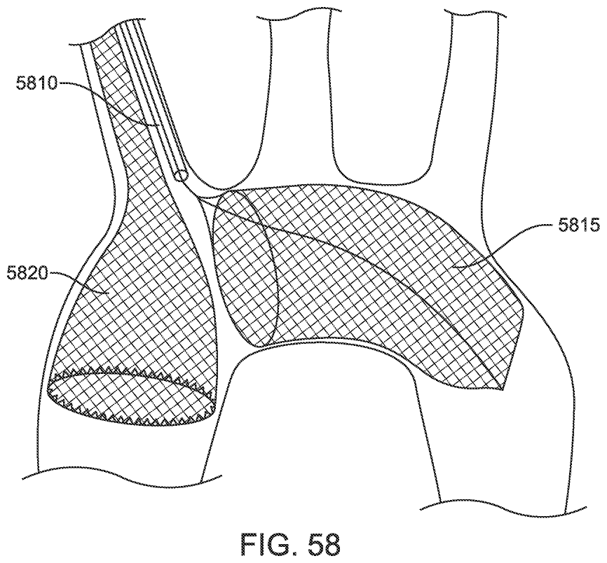

FIG. 58 shows another embodiment of an introducer sheath 5810 that is coupled to a primary filter element 5815 positioned at a distal end of the sheath 5810. A supplementary filter element 5820 serves as a secondary filter that can provide embolic protection to arteries downstream of the primary filter element.

In an embodiment, the filter is at least partially formed of a laser cut Nitinol tube. The laser cut tube may be shape set into a desired shape, such as into a conical configuration. A filter mesh, net or fabric of a desired pore size can be attached to the inner or outer surface of the Nitinol frame to provide embolic protection.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. A system for transcatheter aortic valve treatment, comprising:
an arterial access sheath adapted to be introduced into an access site at the left or right common carotid artery or left or right subclavian artery, wherein the arterial access sheath has a first lumen sized and shaped to receive a valve delivery system configured to deliver a prosthetic valve into a heart or aorta through the arterial access sheath, the first lumen having a first opening at a proximal region of the arterial access sheath and a distal opening at a distal region of the arterial access sheath, first lumen is sized to fit therethrough the valve delivery system;
the valve delivery system, wherein the valve delivery system fits within the first lumen of the arterial access sheath and is configured to deliver a prosthetic aortic valve;
an embolic protection element coupled to the arterial access sheath, the embolic protection element configured to be positioned in an aorta such that the embolic protection element causes blood flow to be redirected away from an orifice of an artery that branches off of the aorta; and
at least one capture filter coupled to the arterial access sheath, the at least one capture filter configured to be positioned in the aorta in a position relative to the embolic protection element.

2. The system of claim 1, wherein the embolic protection element is a filter attached to the arterial access sheath.

3. The system of claim 1, wherein the embolic protection element is a filter that is deliverable through the arterial access sheath.

4. The system of claim 1, wherein the embolic protection element is configured to be at least partially positioned in an ascending aorta.

5. The system of claim 1, wherein the embolic protection element disrupts a blood flow pattern near an orifice of the innominate artery.

6. The system of claim 1, wherein the embolic protection element is configured to be at least partially positioned in an aortic arch.

7. The system of claim 1, wherein the capture filter includes a distal band that extends around a circumference of a distal region of the capture filter.

8. The system of claim 7, wherein the distal band is attached to at least one pull wire.

9. The system of claim 1, wherein the valve delivery system includes:
an inner shaft;
an outer sleeve co-axially aligned with the inner shaft and slidably positioned over the inner shaft;
a valve removably mounted on the inner shaft; and
an actuator coupled to the outer shaft, wherein the actuator can be actuated to retract the outer shaft from a first position to a second position that exposes the prosthetic aortic valve.

10. The system of claim 1, wherein the access sheath further comprises an occlusion balloon at a distal end of the access sheath.

11. The system of claim 1, wherein the embolic protection element causes blood to transition from a laminar flow to a turbulent flow in the aorta.

12. The system of claim 1, wherein the embolic protection element is not a filter.

13. A method of providing embolic protection during an endovascular aortic valve implantation procedure, comprising:
delivering an embolic protection element to an aorta via an access site at the left or right common carotid artery or left or right subclavian artery;
positioning at least a portion of the embolic protection element in the aorta;
causing the embolic protection element to redirect blood flow in the aorta away from an orifice of an artery that branches off the aorta; and
positioning a capture filter in the aorta such that the capture filter captures embolic debris in the aorta.

14. The method of claim 13, wherein the embolic protection element is positioned in an ascending aorta.

15. The method of claim 13, wherein the embolic protection element is positioned to disrupt a blood flow pattern near an orifice of the innominate artery.

16. The method of claim 13, wherein the embolic protection element is delivered to the aorta via an arterial access sheath.

17. The method of claim 16, wherein the embolic protection element is attached to the arterial access sheath.

18. The method of claim 16, wherein the embolic protection element is delivered via an internal lumen of the arterial access sheath.

19. The method of claim 13, wherein the capture filter is positioned downstream of the embolic protection element in the aorta relative to a direction of blood flow.

20. The method of claim 13, wherein the capture filter is positioned upstream of the embolic protection element in the aorta relative to a direction of blood flow.
